(19) United States
(12) Patent Application Publication
(10) Pub. No.: US 2026/0014035 A1
(43) Pub. Date: Jan. 15, 2026

(54) Protective garment

(71) Applicant: AB Lindex, GÖTEBORG (SE)

(72) Inventors: Solgun Drevik, AMBJÖRNARP (SE); Ebba Lo, GUSTAVSBERG (SE); Hasanthi Kaushalya DASANAYAKE MUDIYANSELAGE, MAKOLA (LK); Manoshi Hansapani UBESINGHA ARACHCHIGE, Panadura (LK)

(21) Appl. No.: 19/333,438

(22) Filed: Sep. 19, 2025

(30) Foreign Application Priority Data
Mar. 20, 2023 (SE) 2350311-3

(51) Int. Cl. A61F 13/494; A61F 13/49; A61F 13/496

(52) U.S. Cl. CPC A61F 13/4946; A61F 13/49003; A61F 13/496

(57) ABSTRACT

A washable protective garment includes a front part, a back part, a waist and a crotch part having a leak-proof body. The garment has a dorsal shaping element, including flexible first, second and third element parts. The first element part is fastened to the leak-proof body and is arranged to run along the intergluteal cleft of a standing user whereas the second and third element parts are arranged to extend towards a hip user. The first element part applies a first tension force along the intergluteal cleft of the user, whereas the second and third element parts apply lateral second and third tension forces.

### FIELD OF THE INVENTION

The various embodiments of the present invention relates to a protective garment, such as a pantie, a pair of trousers or other legwear with or without an upper/torso part. The protective garment may also be a skirt, a dress or similar, having a protective crotch part. Specifically, the protective garment is designed to protect against leakage of bodily fluids such as menses, urine and similar when worn by a user. Specifically, the present protective garment may be designed to be suitable for female users.

### BACKGROUND

There are many known solutions to the problem of legwear absorption and leakage protection in various situations.

For instance, GB 2282522 A discloses a sanitary pantie featuring an absorbent body and various elastic elements extending past a crotch part of the pantie to achieve sealing against the skin of the user wearing the pantie, making sure that all bodily fluids are absorbed by the absorbent body.

As another example, WO 2013/148749 discloses a protective pantie provided with an elastic element pulling an integrated sanitary pad upwards against the skin of a user wearing the pantie, so as to achieve adequate contact between the user and the sanitary pad.

WO 2015/177307 A1 and JP 3179880 U disclose garments having separate layers and an absorption body. JP 2017036532 A also discloses a garment with separate layers.

Additional examples of known solutions include WO 2012/047650 A1 and U.S. Pat. No. 10,888,470 B2 describing a washable panty with absorbing material; and WO 2013/147658 A1 and GB 2435403 A, describing panties with separate pads. KR 102165510 B1 and WO 2013143601 A1 also disclose garments intended for use with a separate sanitary napkin.

These solutions may perform acceptably in terms of leakage protection and absorption.

However, there is a need for a protective garment of the general type offering an improved user experience when worn, such as in terms of aesthetic appearance and/or comfort, with maintained absorption and protection against leakage. In particular, there is a need for a washable garment with these properties that does not require a separate sanitary napkin, that can be used repeatedly to provide leakage protection, and be washed in between uses.

### SUMMARY

The present invention solves the above described and other problems.

Hence, the various embodiments of the invention relates to a protective garment in the form of a pantie, a pair of trousers or other legwear with or without an upper/torso part, the protective garment comprising a front part; a back part; a waist; and a crotch part having a leak-proof body, the protective garment being a washable garment arranged for repeated use.

The protective garment comprises a dorsal shaping element, in turn comprising a flexible first element part having a ventral end and a dorsal end, a flexible second element part and a flexible third element part.

The first element part, at its ventral end, is fastened to or constitutes an integrated part of the leak-proof body and is arranged to run along the intergluteal cleft of a standing user wearing the protective garment.

The second and third element parts are each arranged to extend laterally across the waist and/or buttocks of the standing user wearing the protective garment, towards a respective hip of the standing user.

The protective garment is arranged so that, as a result of the standing user wearing the protective garment, the first element part applies a first tension force along the intergluteal cleft of the user whereas the second element part applies an at least partly lateral second tension force and the third element part applies an at least partly lateral third tension force.

The ventral end of the first element part is connected to the second element part via a first force direction transferring connection arranged to distribute the first tension force to the second element part so that the distribution involves a gradual direction change of the distributed force between the first element part and the second element part.

The ventral end of the first element part is connected to the third element part via a second force direction transferring connection arranged to distribute the first tension force to the third element part so that the distribution involves a gradual direction change of the distributed force between the first element part and the third element part.

In some embodiments, the dorsal shaping element is arranged so that the first element part is pressed into the intergluteal cleft of the standing user wearing the protective garment as a result of the first tension force.

In some embodiments, the dorsal shaping element has elastic properties.

In some embodiments, the dorsal shaping element has inelastic properties.

In some embodiments, the dorsal shaping element is less elastic than materials of the protective garment surrounding the dorsal shaping element, at least such materials being located adjacent to the dorsal shaping element on respective longitudinal sides of the first, second and/or third element part.

In some embodiments, an elastic property of the dorsal shaping element is achieved by material of the dorsal shaping element being added via sewing, stitching, knitting, bonding, welding, gluing, infusing; and/or by the material of the protective garment comprising an integrated part of a knitted or weaved textile layer of the protective garment, produced by, during manufacturing, locally varying the properties of the textile layer by changing the weaving or knitting procedure.

In some embodiments, the first element part is tapered, becoming narrower towards the ventral end.

In some embodiments, the ventral end of the first element part has a lateral width of at the most 40 mm, such as at the most 25 mm, and/or at least 10 mm, or even at least 15 mm.

In some embodiments, the second element part and the third element part are both arranged to run laterally to a respective iliac crest of the standing user wearing the protective garment.

In some embodiments, both the first and second force direction transferring connections each is a curved force direction transferring connection arranged to distribute the first tension force in question to the second or third element part, respectively, so that the distribution involves a gradual direction change of the distributed force in question.

In some embodiments, the forced direction transferring connection has a radius of curvature of at least 2.5 cm, or even at least 5.0 cm, for a medium sized protective garment 100 according to EN 13402.

In some embodiments, the back part comprises a respective buttock part, each buttock part being arranged to cover a buttock of the standing user wearing the protective garment, each buttock part comprising a convex bowl shape in a relaxed state of the protective garment, the bowl shape being arranged to at least partly follow a corresponding shape of the buttock of the user wearing the protective garment.

In some embodiments, the protective garment further comprises a dorsal line of a piece of fabric forming part of the protective garment, such as being achieved using a stitched seam, the dorsal line protruding towards a standing user wearing the protective garment along the intergluteal cleft of the user, the dorsal line running at least along, and overlapping with, part of the first element part.

In some embodiments, the dorsal line has a curved shape in a relaxed state of the protective garment, the curved shape being arranged to follow a shape of the intergluteal cleft of the standing user wearing the protective garment.

In some embodiments, the protective garment further comprises a pair of laterally separated shaping elements, each running ventrally from the first element part, in relation to the standing user wearing the protective garment, along either side of the leak-proof body, to a position ventrally of the perineum of the standing user, each of the laterally separated shaping elements being arranged to apply a tension force along its length when the protective garment is worn by the standing user.

In some embodiments, the laterally separated shaping elements are arranged to extend on either lateral side of the labia of the standing user wearing the protective garment, ventrally from the first element part, and arranged so that, when a ventral/upward force is applied to the front part and the back part as a result of the waist being pulled upwards in relation to the user wearing the protective garment, they are in turn forced upwards.

In some embodiments, the laterally separated shaping elements run from the ventral end of the first element part.

In some embodiments, the dorsal shaping element is directly connected to the laterally separated shaping elements.

In some embodiments, the dorsal shaping element, together with the laterally separated shaping elements, forms a “Y”-shape.

In some embodiments, the laterally separated shaping elements run in parallel to the first element part.

In some embodiments, a lateral width of a section of the first element part running in parallel to the laterally separated shaping elements is between 30 mm and 60 mm for a medium sized protective garment according to EN 13402, European standard for Size designation of clothes.

In some embodiments, a respective side part of the protective garment located between the first element part and either of the laterally separated shaping elements is made from a material which is less firm than that of the first element part and less firm than that of the laterally separated shaping element in question.

In some embodiments, the protective garment further comprises a ventral shaping element having a ventral end extending laterally across the lower belly of the standing user wearing the protective garment, the ventral end part being arranged to cause a tension force being applied laterally along the ventral end part as a result of the standing user wearing the protective garment.

In some embodiments, the ventral shaping element extends ventrally from the leak-proof body to the waist.

In some embodiments, the ventral shaping element is tapered so that a ventral end thereof is wider than a dorsal end thereof.

In some embodiments, the protective garment further comprises a friction-increasing layer arranged to abut directly against the skin of the standing user wearing the protective garment, the friction-increasing layer being provided to abut the skin at the lower belly of the user.

In some embodiments, the protective garment further comprises a ventral gripping element, generally laterally elongated and having a convex shape softly bulging ventrally/upwards in relation to the standing user wearing the protective garment, the ventral gripping element being less elastic, in its lateral direction, than materials of the protective garment surrounding the ventral gripping element.

In some embodiments, the protective garment comprises a first pull string and the second element part and a second pull string running along the third element part.

In some embodiments, the first pull string and the second pull string each run also along the first element part.

In other embodiments, the first pull string and the second pull string are both interconnected to a dorsal pull string via a joining member, the dorsal pull string running along the first element part.

In both of these types of embodiments, each of the first, second and the dorsal pull string, if used, can run in respective channels or along respective guides.

In some embodiments, each of the first and second pull strings can be tightened so as to increase the first and/or second tension force.

In some embodiments, the first and/or second and/or dorsal pull string is arranged to be fastened to the protective garment at a point ventrally to the iliac crest of the standing user wearing the protective garment.

In some embodiments, the first and/or second and/or dorsal pull string is arranged to be directly fastened to the leak-proof body or indirectly fastened to the leak-proof body via a connecting part that is less flexible than material laterally surrounding the connecting part.

In some embodiments, the joining member is arranged to run in and along a dorsal channel, the dorsal channel being tapered so that the joining member engages with an inside of the dorsal channel if moved ventrally in the dorsal channel so that the joining member is prevented from moving ventrally in the dorsal channel beyond a certain point.

In some embodiments, the joining member is triangular, fastening to each of the first, second and dorsal pull string in a respective corner of the joining member.

In some embodiments, the first and/or second curved force direction transferring connection is reinforced by a reinforcing element engaging with the first and/or second pull string and forcing the pull strings in a direction laterally towards each other, or holding the pull strings at a minimum lateral distance from each other at the reinforcing element.

In some embodiments, the reinforcing element comprises a rigid element, such as a plastic or metal ring or a local fabric reinforcement, arranged to engage with both the first and the second pull strings and thereby to keep them at a minimum lateral distance from each other at a point near the dorsal end of the first element part.

In some embodiments, the dorsal shaping element is arranged as a separate, overlapping, flexible layer in relation to the rest of the protective garment.

In some embodiments, the dorsal shaping element is non-connected to the protective garment at least between the leak-proof body and a fastening point to the rest of the protective garment.

In some embodiments, the dorsal shaping element is fastened to the leak-proof body and/or runs internally to the leak-proof body when the protective garment is worn by the standing user.

In some embodiments, the dorsal shaping element is fastened to the protective garment at the waist of the protective garment.

In some embodiments, the second element part and the third element part are both be fastened to the protective garment at a respective point ventrally to the iliac crest of the standing user wearing the protective garment, so that each of the second element part and the third element part runs, from the dorsal end of the first element part, around the iliac crest of the standing user wearing the protective garment and again laterally towards but not up to a front center line of the front part.

In some embodiments, the dorsal shaping element is fastened to a dorsal end of the leak-proof body.

In some embodiments, the dorsal shaping element is fastened to a ventral end of the leak-proof body, and runs over the leak-proof body, internally to the leak-proof body when the protective garment is worn by the standing user, between a dorsal end of the leak-proof body and the ventral end of the leak-proof body.

In some embodiments, a section of the dorsal shaping element running across the leak-proof body is tapered so that a dorsal end of the section is narrower than a ventral end of the section, in relation to the standing user wearing the protective garment.

In some embodiments, a section of the dorsal shaping element running across the leak-proof body is laterally narrower than the leak-proof body.

In some embodiments, at least the section of the dorsal shaping element running across the leak-proof body comprises at least one of a liquid-absorbing layer and a wicking layer.

In some embodiments, at least the section of the dorsal shaping element running across the leak-proof body comprises a liquid-barrier layer.

In some embodiments, the protective garment comprises a ventral shaping element.

In some embodiments, the ventral shaping element is fastened to the leak-proof body.

In some embodiments, the ventral shaping element is fastened to the protective garment at the waist of the protective garment but is non-connected to the protective garment between the waist and the leak-proof body, or the ventral shaping element is connected to the dorsal shaping element near the waist.

In some embodiments, the ventral shaping element is fastened to the leak-proof body at a dorsal and/or ventral end of the leak-proof body.

In some embodiments, at least a section of the ventral shaping element running across the leak-proof body comprises at least one of a liquid-absorbing layer and a wicking layer.

In some embodiments, at least the section of the ventral shaping element running across the leak-proof body comprises a liquid-barrier layer.

In some embodiments, the first element part is fastened to the leak-proof body at the perineum of the standing user wearing the protective garment.

In some embodiments, the dorsal shaping element constitutes, is comprised in or comprises a joining area, joining together the leak-proof body, such as a liquid-absorbing layer comprised in the leak-proof body, to the back part.

In some embodiments, the leak-proof body has a laterally local narrowest part arranged between the legs, such as at the gracilis, of the standing user wearing the protective garment.

### BRIEF DESCRIPTION OF THE DRAWINGS

FIG. 1a shows a first protective garments in a perspective view, in accordance with some embodiments;

FIG. 1b is a flattened view of the first protective garment, in accordance with some embodiments;

FIG. 1c is a detail flattened view of a dorsal shaping element, in accordance with some embodiments;

FIG. 1d is a flattened view of a second protective garment, in accordance with some embodiments;

FIG. 2 is a flattened view of a third protective garment, in accordance with some embodiments;

FIGS. 3a-3c are respective flattened views of a fourth, fifth and sixth protective garment, in accordance with some embodiments;

FIG. 3d is a detail section view, taken in a vertical cross-section, of a crotch part, in accordance with some embodiments;

FIG. 4 is a flattened view of a seventh protective garment, in accordance with some embodiments;

FIGS. 5a-5l are respective perspective views of eighths, nineths, tenths, elevenths and twelfths protective garments, in accordance with some embodiments;

FIGS. 6a-6g are respective section views, taken in a vertical cross-section, of different layer arrangements of a thong-like section and a leak-proof body, in accordance with some embodiments;

FIG. 7a is a flattened view of a thirteenth protective garment as seen from an outside, in accordance with some embodiments; and

FIG. 7b is a flattened view of the thirteenth protective garment as seen from an inside, in accordance with some embodiments.

It is noted that the Figures illustrate different exemplifying embodiments of the present invention. These embodiments have been selected in order to clearly illustrate various aspects and features described herein, and it is understood that individual features and properties illustrated in different Figures can be combined in any way, subject to compatibility.

It is further noted that the Figures share the same reference numerals for same and corresponding parts. The Figures are intended to illustrate the principles used by the various embodiments of the present invention, and are not necessarily to scale.

It is further noted that the present solutions circle around a number of different cooperating features that can be combined in various ways so as to solve the initially described problem. This will become clear from the following description of a number of embodiment examples of the present invention.

### DETAILED DESCRIPTION

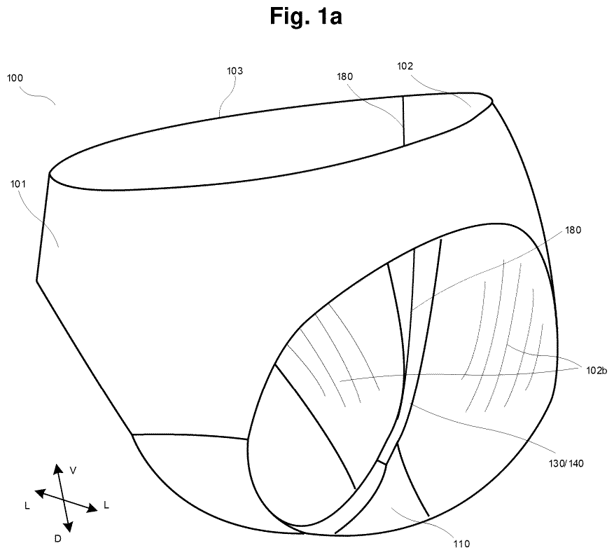

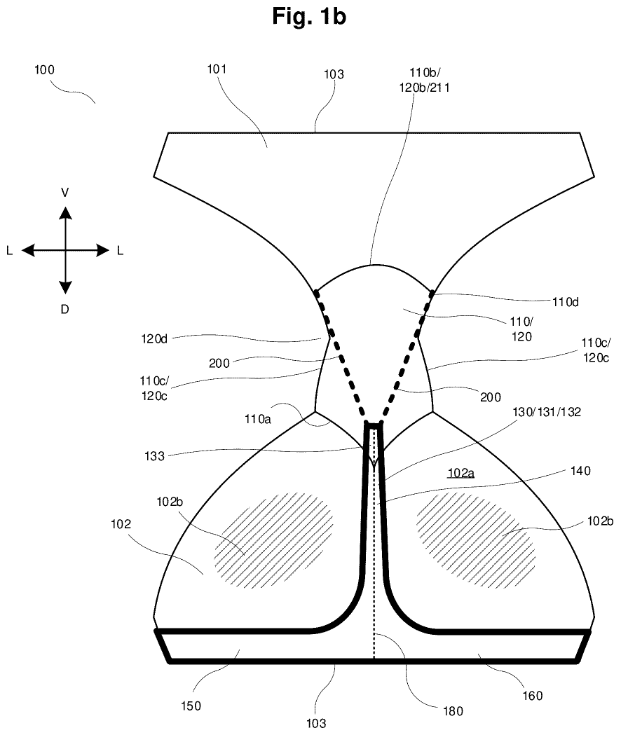

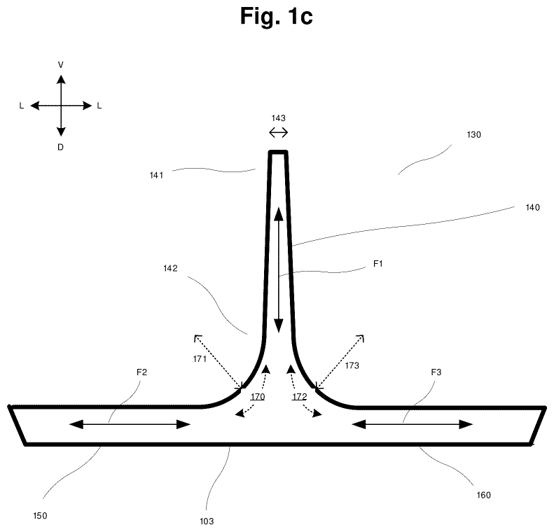

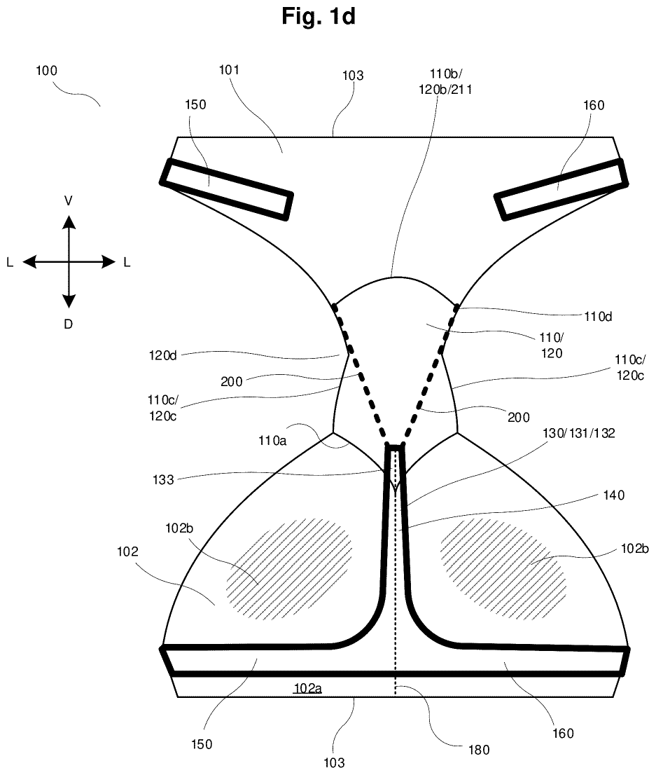

Hence, FIG. 1a shows a protective garment 100 according to one embodiment of the invention in a perspective view, while FIG. 1b shows the same protective garment 100 in a flattened view.

In FIG. 1b, lateral directions L are shown, as well as a dorsal direction D and a ventral direction V. These directions apply correspondingly to the other Figures, as applicable. Furthermore, U denotes the upwards direction, in relation to the standing, upright user wearing the protective garment 100.

Even though the present protective garment 100 can be intended both for male and female users, some of the embodiments will work particularly well for female users. Hence, for all embodiments described herein a male or a female user is intended, but the protective garment 100 can also be specifically intended for a female user.

The protective garment 100 is in the form of a pantie, a pair of trousers or other legwear, such as tights, leggings, shorts or a skirt with integrated panty. Furthermore, the protective garment 100 can be designed with or without an upper/torso part. For instance, the protective garment 100 can be a body with integrated panty, or a jumpsuit. Moreover, the protective garment 100 can be a skirt, a dress, a piece of active wear, a piece of shapewear, or a swimwear. Of course, it is also possible that the protective garment 100 is of more than one of these exemplifying types at once, such as a swimwear in the form of a skirt.

The protective garment 100 comprises a front part 101, a back part 102, a waist 103 and a crotch part 110. It is understood that these parts 101, 102, 103, 110 may each be separate and joined-together parts of the garment 100, or that two or more of these parts 101, 102, 103, 110 may be integrated. Hence, some of these parts 101, 102, 103, 110 may be integrated and the garment 100 may be manufactured by joining together such separate and/or integrated parts so as to form the complete protective garment 100. For instance, the front part 101 and the back part 102 may be two areas (subparts) of one single integrated part, being joined together with a separate crotch part 110, or the front part 101 and the back part 102 may constitute two separate but joined together parts, the crotch part 110 then forming a third separate but joined-together part. These are only a few of many different possible examples of how to construct a protective garment of the present type based on a number of different joined-together subparts.

Put differently, the crotch part 110 may be a separate part from the front part 101 and/or from the back part 102. Concretely, the front part 101 and the back part 102 may for instance be manufactured as a tube-shaped textile, after the crotch part 110 joins them together at the groin to form the protective garment 100. In some embodiments, the crotch part 110 can form an integrated part of the front part 102, and a join between the crotch part 110 and the back part 102 can be arranged to also form a join between the crotch part 110 and the dorsal shaping element 130, the latter join being as in any of the ways described below, such as forming a “V” shape having an angle pointing in the dorsal direction.

As will be clear from the below, each of the parts 101, 102, 103, 110 may also in itself comprise several separate and joined together parts. For instance, the crotch part 110 may comprise several layers being joined together. Each of such layers may, in turn, overlap or not overlap with the front part 101 and/or back part 102.

The waist 103 may be a separate part, joined together with the front part 101 and the back part 102, or be an integrated subpart of the front part 101 and/or back part 102.

The protective garment 100 can also comprise additional separate or integrated parts, such as the below-described dorsal shaping element 130.

In general, different separate parts of the protective garment 100 may be permanently joined together using one or several joining techniques that may be at least partly conventional as such, such as using stitching, knitting (such as flat knitting), welding (such as ultrasonic welding) and/or gluing.

One example of such joining techniques is bonding. One example of bonding is silicone bonding. Here, silicone is applied, for instance using a screen print process, to a surface of a first layer, such as to form a glue line or a particular desired pattern. Then, a second layer is arranged on top of the first layer, and the two layers are glued together by the silicone, such as by application of pressure and/or heat. Of course, other gluing substances with corresponding properties can be used instead of silicone, as the case may be. The bonding can be performed using a tool having a desired pressing pattern and/or shape to achieve a joint having particular properties, such as a 3D shape in a relaxed state of the protective garment 100.

Another example is textile welding, whereby heat is applied, such as via hot air or a wedge, to at least two layers arranged on top of each other, welding together the layers using melting along a join line or pattern. As an alternative to heat, ultrasonic energy can be delivered to the welded substrate.

Another example is stitching, that can be conducted using many different types of seams and with different types of one or several threads. For instance, seam types and/or thread types can be selected to achieve local constrictions in a sewn knitted, woven or non-woven material, and/or affect elasticity, stiffness or a shape of the material being stitched. Apart from stitching, both gluing and bonding can also be used to affect 3D properties of the final garment, such as elasticity and stiffness in different directions, and also to impart a certain preformed shape upheld using the glue, bond or seam. These different bonding methods can also be combined in any one produced piece of protective garment 100, depending on requirements and desired properties.

In addition, or alternatively, to such 3D shaping by bonding, different ones of the parts 101, 102, 103, 110 may also be shaped using seamless techniques, for instance by varying a knitting pattern and/or a knitting needle used and/or a thread used across the surface of such a part 101, 102, 103, 110. In addition, thereto or alternatively, the layers forming one or several of the parts 101, 102, 103, 110 may be varied, with respect to material used and/or internal bonds, to achieve similar results.

Similarly, individual ones of the parts 101, 102, 103, 110 and/or separate elements such as the below-described dorsal shaping element 130 or ventral shaping element 210 can be provided with (or arranged in the form of, in the case of the separate elements) lines or patterns of applied silicone or other glues, welds and/or stitches to form features having desired shapes and properties. For instance, such desired properties may include a desired local flexibility, dumbness, firmness or elasticity. As a concrete example, the dorsal shaping element 130 may be provided as an application of an elongated silicone area, stitch line or weld line on the back part 102 and/or on the crotch part 110, that may or may not include a separate layer of textile or other flexible material fastened to the back part 102 and/or the crotch part 110 using the bond, stitch or weld. Correspondingly, the ventral shaping element 210 may be provided as an application of an elongated silicone area, stitch line or weld line on the front part 101 and/or on the crotch part 110, that may or may not include a separate layer of textile or other flexible material fastened to the back part 101 and/or the crotch part 110 using the bond, stitch or weld.

Hence, glue, bond, a weld, a knit and/or a seam may be applied to a flexible element without joining the element in question to another element, in other words only to affect the local material properties of the element in question.

Furthermore, such gluing, stitching, knitting and welding can be tailored with specific liquid permeability properties. For instance, a hydrophobic glue or stitching thread, or a liquid-tight weld using completely melted plastic textile material can be arranged to constitute a liquid barrier, preventing liquid from spreading laterally across joined together textile sheets past the joint in question.

More than one of the joining and application techniques may also be combined, such as stitching a glued joint or providing a layer of silicone on top of two stitched together panels of textile material, along a stitch line joining the two panels together. Various joining and applications may also be combined in different layers, having different lateral extensions. For instance, a pattern formed by silicone application may be arranged on top of a layer that is in turn joined to a different layer using stitching, welding or bonding.

As mentioned, surfaces and/or the interior structure of one or several layers of one or several of the parts 101, 102, 103, 110 may furthermore be treated to achieve desired properties without being locally joined at the place of treatment to an additional layer. For instance, fiber flocking can be used, where fibers of plastic material are permanently fastened, such as standing or in random orientations, to the surface in question. Such flocking can be made to achieve a soft surface that is agreeable when in direct contact with the skin of a user and/or affect other mechanical properties such as elasticity or surface friction.

In a different example, molten plastic material can be injected into the layer in question and be allowed to set therein, altering the mechanical properties such as elasticity and/or stiffness and/or surface friction of the layer in question. One example of the latter is so-called “liquid lycra” processing.

Another example is the above-discussed surface application of silicone or other compounds, for instance compounds that may in some cases also be used as bonding agents. Such application can locally affect elasticity, stiffness, surface friction, shape and other mechanical properties of the surface to which the application is performed. For instance, silicone can be applied using printing or extrusion, the latter alternative in a process called “silicone extrusion” in which silicone is extruded via a nozzle. Such printed or extruded silicone, that is then present on a surface of the fabric, is capable of producing a 3D shape on the surface of the fabric, can also be flocked with fibers that are fastened to the applied silicone. An additional example is “surplus penetration”, where silicone is printed or otherwise applied onto a fabric surface, or between two layers of fabric surface, so as to penetrate into the material, providing both a desired elasticity along the printed areas and a high-friction surface of the fabric and/or bonding together of the two layers.

In the various examples provided herein, flocked fibers may be fastened using silicone, or any other suitable adhesive, as a binder. Useful flock adhesives include single-part and two-part catalyzed systems, and both plastisol and water-based adhesives can be used. Various useful silicone adhesives are described in US2021246312 A1. As one of many alternatives, a water-based acrylate coating adhesive is described in CN 112794938 A.

Seamless knitting, such as tubular knitting or flat-knitting, can also be used to produce parts or all of the present protective garments. In this case, the knitting may be locally varied so as to locally affect mechanical properties, such as elasticity, for instance by introducing local constrictions or similar.

As an alternative to a flocked surface, a velvet, velour or plush material, either elastic or inelastic, can generally be used in the examples described herein.

Hence, various techniques of the above-discussed type can be used to produce the various features described herein, by locally affecting mechanical properties of the different parts 101, 102, 103, 110. The same and/or different techniques can also be used to join various material layers and/or parts 101, 102, 103, 110 together. Sometimes, a joining technique may also affect the mechanical properties in a desired manner, and sometimes a particular joining technique can be combined with a particular application technique as mentioned above to locally achieve a particular set of desired mechanical properties.

The various parts and elements 101, 102, 103, 110, including their individual subparts, of the protective garment 100 described herein may be manufactured with any combination of one or several of the above-described application and/or bonding methods, as the case may be, to achieve the mechanical properties described below in connection to each such element or part.

As mentioned, seamless knitting can be used to produce the protective garment 100. For instance, the whole garment 100, or one or several of the parts 101, 102, 103, 110, can be produced by seamless knitting, without any seams, bonds or welds. Then, the garment 100 may comprise various layers as described herein, while at least one such layer is common for all or at least several of the parts 101, 102, 103, 110, while various parts 101, 102, 103, 110 may comprise additional layers. By joining together the various layers, the final garment 100 is then assembled. In a different example, tubular knitting is used in combination with a joining together of the front part 101 to the back part 102 at or via the crotch part 110 (or leak-proof part 12) to form the final garment 100, with or without additional layers such as described herein.

In particular, in some embodiments at least one layer of the crotch part 110 is manufactured from the same integrated flexible material as at least one layer of the front part 101 and/or at least one layer of the back part 102. A peripheral shape of the at least one layer of the crotch part 110 is then defined, in relation to the at least one layer of the front part 101 and/or of the back part 102, by areas of varying stitching types, stitching, flocking, gluing and/or welding.

As used herein, the term “integrated material” denotes a body of knitted, woven or non-woven flexible material that has not been joined together from separate subparts, such as using any of the joining techniques described above, but has instead been produced as one connected piece of flexible, knitted, woven or non-woven material by weaving, stitching or some other technique for producing such material. For instance, non-woven materials may be staple non-wovens, melt-blown non-wovens or spunlaid non-wovens. Correspondingly, an “integrated part” is a part being formed as a part of the same, connected material body as it is a part of.

As the term is used herein, a “non-woven” material is a fabric-like material made from short and/or long fibers, for instance multifilament fibers, that are bonded together, such by chemical, mechanical, heat or solvent treatment. One example of such material with short or long fibers could be felt. A non-woven material is neither woven nor knitted.

All which has been said above in relation to the manufacture, properties and fastening together of parts 101, 102, 103 and 110 also correspondingly applies to the other distinct parts described herein, such as the various parts 140, 150, 160 of a dorsal shaping element 130; and a ventral shaping element 210 (see below). This is in particular true in case the dorsal shaping element 130 and/or the ventral shaping element 210 is or are formed as separate-layer structures in relation to, for instance, the parts 101, 102 and/or 110.

The waist 103 may comprise a waist band, which may in turn be formed with desired elastic and friction properties, for instance by application of silicone to the surface of the waist near and/or at an upper edge of the waist 103, by a silicon bond of two material layers or infusion.

This silicone can also form a protection against fraying. The corresponding technique can be used to form leg opening edges of the garment 100.

The description herein uses a standing user wearing the protective garment 100 as a frame of reference for the various parts and directions of the garment 100. Hence, the geometry of the user's anatomy (for instance, “dorsal” and “ventral”) and/or an external coordinate system (such “up” or “down”) are used to characterize various geometric aspects.

As mentioned, the user is typically a female user. However, it is understood that the present principles can also be applied for garments intended for male users, as applicable.

The crotch part 110 comprises a leak-proof body 120. In many cases, the leak-proof body 120 in turn comprises or is constituted by a liquid-absorbing layer 124, arranged to absorb and hold liquid released onto the leak-proof body, such as a user wearing the protective garment 100. Furthermore, the leak-proof body 120 can comprise a liquid barrier 126, such as a plastic film, arranged to prevent leakage of liquid through the leak-proof body 120 and out from the protective garment 100 when worn by a user. In some cases, the leak-proof body 120 may contain no liquid-absorbing layer, but provide its leak-proofness only via a liquid barrier 126. The liquid barrier 126 in itself may be in the form of or comprise a flexible fabric having a liquid-proof-treated coating or surface; a liquid-impermeable (such as hydrophobic and/or impregnated) fabric and/or a plastic film. In specific examples, the liquid barrier 126 may comprise a knitted, woven or non-woven material which is in itself hydrophobic, such as a so-called spunbond-meltblown material, comprising one or several meltblown layers sandwiched between a pair of spunbonded layers. In other words, the liquid barrier 126 may be arranged to be penetrable to water vapour (to “breathe”), but at the same time be impermeable to liquids. Of course, the liquid barrier 126 may in itself comprise several layers, each having one or several of the different properties. The corresponding applies to the barriers 148 and 217 (see below).

In general terms, the crotch part 110 is a part arranged at the crotch of the user wearing the protective garment 100.

Also on a general note, the leak-proof body 120 can be a part arranged to block, when the garment 100 is worn by the user, liquid leakage by being liquid impermeable and/or absorbing.

As is illustrated in FIGS. 1a-1d, the protective garment 100 can comprise a dorsal shaping element 130.

As used herein, the term “shaping element” refers to a separate or integrated part of the garment 100 arranged to locally affect the shape of the garment when a standing user wears the protective garment 100, and in particular as a result of a shaping of the garment 100 being a consequence of the wearing of the garment 100 (as opposed to when the garment 100 is not worn but instead, for instance, lays freely on a surface). In other words, the presence of the shaping element alters the shape of the protective garment 100 as it is worn by the standing user in a way so that the shape is different, and in particular functionally different, as compared to the hypothetical case in which the garment 100 is without the shaping element in question.

The dorsal shaping element 130 can be arranged along the intergluteal cleft of the standing user wearing the protective garment 100. It can be fastened to or comprise an integrated part of the back part 102, or form a separate part in relation to the back part 102 as is described in closer detail below.

The dorsal shaping element 130 as a whole can comprise or be completely made from flexible material.

The dorsal shaping element 130 can comprise a flexible first element part 140, in turn having a ventral end 141 and a dorsal end 142. The dorsal shaping element 130 can also comprise a flexible second element part 150 and a flexible third element part 160.

The dorsal shaping element 130, and in particular the first element part 140, can be fastened to or constitute an integrated part of the leak-proof body 120, in particular at its ventral end 141. In other words, the ventral end 141 can constitute or form part of a join between the first element part 140 and the leak-proof body 120.

Each of the first element part 140, the second element part 150 and the third element part 160 can be of generally elongated shape. The first element part 140 can be arranged to run in the medial plane of the standing user wearing the protective garment 100. The second element part 150 and the third element part 160 can each be arranged to run laterally outwards, in opposite lateral directions along the back parts of the user, in relation to the standing user wearing the protective garment 100 from the dorsal end 142 of the first element part 140, out to a hip of the user. Each of the second element part 150 and the third element part 160 can furthermore continue past the hip of the user, and run laterally inwards, again in laterally opposite directions, along the front parts of the user, such as the user's belly.

Hence, the second 150 and third 160 element parts can each be arranged to extend laterally (with at least a lateral component, the lateral component of extension preferably being larger than a vertical component of extension for the element part 140, 150, 160 in question) across the waist and/or buttocks of the standing user wearing the protective garment 100, towards, possibly up to and possibly even past a respective hip of the standing user.

In general, the second element part 150 and the third element part 160 can both be arranged to run laterally (with the corresponding meaning), from the first element part 140, to, and possibly past, a respective iliac crest of the standing user wearing the protective garment 100.

The second 150 and third 160 element parts can be joined or meet at the front side of the standing user wearing the protective garment, such as on the front part 101, or end at a distance from each other, not meeting. In a special case, the second 150 and third 160 element parts form part of, or even constitute, the waist 103. However, in some embodiments the second 150 and third 160 element parts exist independently of the waist 103, for instance by being formed as independent pieces of flexible material that are not directly fastened or connected to the waist 103 at all or that are only fastened to the waist 103 along part of the waist 103 or only at one or several discrete points along the waist 103, and/or by the second 150 and third 160 element parts being formed below and at a vertical distance from the waist 103 when the protective garment is worn by the standing user. It is understood that the material forming the rest of the protective garment 100, such as textile material forming the front part 101 and/or the back part 102, can be fastened to both the waist 103 and to the dorsal shaping element 130 while still separating these elements one from the other so that they are not directly fastened or connected one to the other.

FIG. 1c is a detail view of the dorsal shaping element 130 illustrated in FIG. 1b. FIG. 1c illustrates tension forces F1, F2, F3 occurring in the dorsal shaping element 130 as a result of the standing user wearing the protective garment 100. It is hence understood that these tension forces F1-F3 do not occur when the protective garment 100 is in a relaxed state; when it lies on a surface instead of being worn; or similar.

The protective garment 100 can hence be arranged so that, as a result of the standing user wearing the protective garment 100, the first element part 140 applies or gives rise to a first tension force F1 along the intergluteal cleft of the user; whereas the second element part 150 applies or gives rise to an at least in part laterally acting second tension force F2 and the third element part 160 applies or gives rise to an at least in part laterally acting third tension force F3.

It is understood that the first tension force F1 occurs along the first element part 130, and changes direction along the intergluteal cleft of the standing user while remaining in the median plane of the user.

It is also understood that each of the second tension force F2 and the third tension force F3 occurs along the second 150 and third 160 element part, respectively, and changes direction while following the path of the element part 150, 160 in question along the anatomic shape of the standing user's back parts.

The protective garment 100 can be arranged so that, when the standing user wears the protective garment 100, the first tension force F1 occurs along the entire length of the first element part 140; the second tension force F2 occurs along the entire length of the second element part 150; and the third tension force F3 occurs along the entire length of the third element part 160.

The combination of the tension forces F1, F2, F3 forces the first element part 130 into the intergluteal cleft of the standing user, much like a conventional thong does. The present inventors have discovered that by combining features achieving concurrently the forces F1, F2 and F3 in one and the same protective garment 100 achieves a very snug, comfortable and secure fit for a broad range of washable garment types, also when the user moves about in various ways.

The ventral end 141 of the first element part 140 can be connected to the second element part 150 via a first force direction transferring connection 170, arranged to distribute the first tension force F1 to the second element part 150. In other words, the first tension force F1 affects the second element part 150 so that the second tension force F2 is increased by the existence of the first tension force F1, this effect being mediated by the first force direction transferring connection 170. Correspondingly, the first force direction transferring connection 170 can distribute the second tension force F2 to the first element part 140, so that the existence of the second tension force F2 increases the first tension force F1.

The distribution can then involve a gradual direction change of the distributed force between the first element part 140 and the second element part 150. This gradual direction change can be achieved in many different ways, illustrated in FIG. 1c as a smoothly curving interconnection between the first element part 140 and the second element part 150. It is noted that in the example illustrated in FIG. 1c, the first element part 140, the second element part 150 and the third element part 160 are in fact subparts of one and the same connected structure, that in turn may be provided using any of the above-described methods, such as seamless knitting or silicon extrusion.

In a way that corresponds to what has been said above in relation to the first element part 140 and the second element part 150, the ventral end 141 of the first element part 140 can be connected to the third element part 160 via a second force direction transferring connection 172, arranged to distribute the first tension force F1 to the third element part 160, in some embodiments so that the distribution involves a gradual direction change of the distributed force between the first element part 140 and the third element part 160.

Hence, when the standing user wears the protective garment 100, pulling the garment 100 upwards in the process of putting it on, the tension forces F1, F2, F3 will result as a result of the wearing. The force F1 may at least partly occur as a result of the waist 103 being pulled upwards. The forces F2, F3 may at least partly occur as a result of a fit of the protective garment 100 along the waist of the user, such that the fabric of the protective garment 100, and as a result also the second 150 and third 160 element parts, along a line encircling the wearing user in a transverse plane of the user is stretched as a result of the protective garment 100 being worn.

In the example illustrated in FIG. 1b, the first element part 140 comprises a seam or joining area, interconnecting the back part 102 and the crotch part 110, while extending some ways dorsally from a dorsal end edge 110a of the crotch part 110 along the back part 102. Hence, in this example the dorsal shaping element 130 constitutes a separate part (the seam or joining material itself), while interconnecting the back part 102 and the crotch part 110. It is realized that the back part 102 and the crotch part 110 can also be connected in additional ways, such as along a lateral joining line that may not be part of the dorsal shaping element 130. Such joining may involve one or several layers of the back part 102 and/or the crotch part 110. The seam forming the first element part 140 in this example may be sewn to create a local constriction of the fabric, and the stitching technique and/or thread may be selected so that the constriction is arranged to locally provide an increased elasticity and/or stiffness of the fabric. This can correspondingly be achieved by a joining area comprised in or constituting the first element part 140.

The second 150 and third 160 element parts can be formed from the same or different techniques as compared to the first element part 140. The shape, such as a gradual curve, of each of the force direction transferring connection 170, 172 can be achieved by the area of application of such techniques describing the shape in question.

In general, the dorsal shaping element 130 may comprise a part 132 being separate from (but permanently fastened to) the crotch part 110 and the back part 102, and the separate part 132 can then in some embodiments connect the crotch part 110 to the back part 102.

As an alternative or supplement, any of the parts 140, 150, 160 forming the dorsal shaping element 130 can comprise a respective elongated constriction in the seamless-stitched material of the garment 100, and in some embodiments where the back part 102 and the crotch part 110 are integrated parts in relation to each other. The dorsal shaping element 130 can then run across both the back part 102 and the crotch part 110. The constriction can be arranged to pull together the fabric locally so as to achieve a locally increased tension in the fabric when the fabric is extended along its surface, in particular when it is pulled apart in a dorsal-anterior direction (for the first element part 140) and/or in a lateral direction (for the second 150 and third 160 element parts). The constriction can also be arranged to locally provide a locally increased elasticity and/or stiffness of the fabric.

Generally, any or all of the parts 140, 150, 160 of the dorsal shaping element 130 can comprise a respective integrated part 131, that may constitute the entire shaping element 130 or only one or several of the parts 140, 150, 160 of the shaping element 130, of a knitted or weaved textile layer of the protective garment 100. Such a knitted or weaved textile layer can then be produced by, during manufacturing, locally varying the properties of the textile layer by changing the weaving or knitting procedure, as discussed above.

Furthermore, the back part 102 can be joined to the crotch part 110 using a scam or a glue/weld bond, while any or all of the parts 140, 150, 160 of the dorsal shaping element 130 can be applied in the form of a patterned elongated silicone application on the surface of a respective layer of the back part 102, also extending across the surface of a layer of the crotch part 110. The silicone can then be arranged to locally provide an increased elasticity and/or stiffness of the fabric, and can be arranged on an inner surface or on an inner layer so that it is not visible externally on the protective garment 110. Any or all of the parts 140, 150, 160 of the dorsal shaping element 130 can comprise a flocked area, with corresponding properties. This flocked area can be a part of a larger set of flocked areas of the different types described herein, such as any “Y”-shape and/or the ventral shaping element 210 and/or the ventral gripping element 230 discussed below. In particular, such a flocked first element part 140 can extend outside of the crotch part 110 and/or outside of the leak-proof body 120, along the back part 102. The back part 102 can also be joined to the crotch part 110 in other ways, such as gluing with other gluing substances than silicone. The corresponding can also apply to the ventral shaping element 210 and/or the ventral gripping element 230 with respect to the crotch part 110, the leak-proof body 120 and the front part 101. The ventral shaping element 210 and/or the crotch part 110 could also, for instance, be fastened together with the front part 101 using “surplus penetration”.

In other embodiments, the crotch part 110 can be shaped with a dorsally elongated extension, overlapping with the back part 102, the elongated extension being fastened to the back part 102 by a suitable seam or glue/weld joint and constituting part or all of the first element part 140. The overlap and/or the joining itself can be arranged to locally provide an increased elasticity and/or stiffness of the fabric. It is understood that the dorsally elongated extension can then comprise all layers of the crotch part 110 or of the leak-proof body 120, or only some of the layers, such as the liquid-absorbing layer 124 and/or the liquid barrier layer 126 (see below).

As have been made clear above with respect to combinations in general, these various examples of how to provide the material properties of the first 140, second 150 and third 160 element parts may be combined in different ways.

The pressing into the intergluteal cleft of the first element part 140 in relation to the standing user wearing the protective garment 100 can be achieved in different ways.

In some embodiments, the dorsal shaping element 130, and in particular the first element part 140, has elastic properties at least along a respective main longitudinal direction of the element part 140, 150, 160 in question. In such cases, the dorsal shaping element 130, and in particular the first element part 140, can be less elastic than materials of the protective garment 100 surrounding the dorsal shaping element 130 (the first element part 140), at least as compared to such materials being located adjacent to the dorsal shaping element 130 on respective longitudinal sides of the first 140, second 150 and/or third 160 element part in question. This difference in elastic properties hence then gives rise to the first tension force F1 and directly or indirectly also to the second F2 and third F3 tension forces.

In other embodiments, the dorsal shaping element 130, and in particular the first element part 140, has inelastic properties at least along a respective main longitudinal direction of the element part 140, 150, 160 in question. Then, the tension forces F1, F2, F3 can accrue as a result of such inelastic properties in a way corresponding to the former case, further as a result of the fabric of the protective garment 100 in general is to certain degree elastic.

As exemplified above, a desired elastic property of the dorsal shaping element 130 and any or all of its parts 140, 150, 160 can achieved by material thereof being added via sewing, stitching, knitting, bonding, welding, gluing, infusing during manufacture of the protective garment 100. In such and other embodiments, a desired elastic property of the dorsal shaping element 130 and any or all of its parts 140, 150, 160 can be achieved by material of the protective garment 100 comprising an integrated part 131 of a knitted or weaved textile layer of the protective garment 100, produced by, during manufacturing, locally varying the properties of the textile layer by changing the weaving or knitting procedure.

As will be exemplified below, the dorsal shaping element 130 can also in part or entirely be arranged as a separate part in relation to the rest of the protective garment 100, only connected at one or more locations but otherwise arranged to move freely in relation to the rest of the protective garment 100.

As illustrated in FIG. 1b, the second 150 and third 160 element parts can connect to, or even constitute part or the whole of, the waist 103. In contrast thereto, and as is illustrated in FIG. 1d, the second 150 and third 160 element parts can end dorsally below the waist 103, before reaching the waist 103. Then, the waist 103 can form a part of the protective garment 100 that is at least partly separated from the second 150 and third 160 element parts by fabric not forming part of the waist 103 nor the dorsal shaping element 130.

FIG. 1d also show an example of the second 150 and third 160 element parts continuing ventrally past the hip, up to a respective lateral end point located on, such as fastened to or at least allowing the respective force F2, F3 to act in relation to, the front part 103, such as on the lower belly of the standing user wearing the protective garment 100.

In general, the back part 102 can comprise a panel part 102a, separating the dorsal shaping element 130, or at least the first element part 140, from the waist 103, such as an upper end edge of the waist 103, so that the dorsal shaping element 130, or at least the first element part 140, does not extend all the way up to the waist 103 (or does not extend all the way up the upper end edge of the waist 103). The panel part 102a can in its entirely be made of flexible, non-stiff knitted, woven or non-woven material. In various embodiments, the dorsal shaping element 130, or at least the first element part 140, may be separated from the waist 103, or from the end edge of the waist 103, by at least 2 cm, such as at least 5 cm, for a medium size of the garment 100 according to EN 13402, European standard for Size designation of clothes. Such a medium size corresponds to a waist girth of 76-80 cm.

It is understood that in case the garment 100 comprises an upper or torso part, there may be no end edge of the waist 103. It is, however, preferred that even in this case the waist 103 is defined as an elastic part running around the waist of the user wearing the garment 100. Even if there are other per se known ways to hold a garment 100 of the present type up, without moving downwards as the wearing user moves around, having a waist 103 apply a certain tension around the waist or hips of the user is a simple way to achieve this.

The dorsal shaping element 130 can be arranged so that, when a dorsal/upward force is applied to the dorsal shaping element 130, and in particular to the first element part 130, via the panel part 102a of the back part 102 and/or via the second 150 and third 160 element parts, and potentially as a result of the waist 103 being pulled upwards in relation to the user wearing the protective garment 100, it in turn forces a section, such as a laterally central section, of the crotch part 110, extending along the intergluteal cleft of the user, in an upwards direction. In some embodiments, at least a central part of the dorsal end edge 110a, 120a of the crotch part 110 and/or the leak-proof body 120 is forced upwards this way.

By forcing the crotch part 110 upwards in relation to the standing user wearing the protective garment 100, a dorsal part of the crotch part 110 can be pressed into the intergluteal cleft of the user, providing a tighter fit.

Hence, both part of the crotch part 110 and the first element part 140 can be arranged to be pressed into the intergluteal cleft of the standing user wearing the protective garment. However, in other embodiments, that will be detailed below, the crotch part 110, and in particular the leak-proof body 120, is not pressed into the intergluteal cleft of the wearing user, but only the first element part 140.

As mentioned above, the first element part 140 can be fastened to the leak-proof body 120 in connection to the dorsal end edge 120a thereof and/or at other locations along the leak-proof body 120. It is generally preferred that the first element part 140 runs up to a fastening point to the leak-proof body 120 arranged at the perineum (the inflection point of curvature) of the standing user wearing the protective garment 100. This does not rule out that the first element part 140 is fastened to the leak-proof body 120 in other locations also, but the first tension force F1 preferably acts, when the standing user wears the protective garment 100, to pull the leak-proof body 120 dorsally via the fastening point at the perineum.

As is shown in FIG. 1b, the first element part 140 can be tapered, so that it becomes narrower towards its ventral end 141. In some cases, the first element part 140 is continuously narrowing towards the ventral end 141, and/or the ventral end 141 is the laterally narrowest part of the first element part 140.

In some embodiments, the ventral end 141 of the first element part 140 has a lateral width 143 (see FIG. 1c) of at the most 40 mm, such as at the most 35 mm, or at the most 30 mm, or even at the most 25 mm. In these and other cases, the ventral end 141 can be at least 10 mm, or even at least 15 mm. In these and other cases, the ventral end 141 can be attached along its entire lateral width 143 to the leak-proof body 120. In case the first element part 140 comprises a part arranged to mediate the first force F1, such as a strap running in a channel or along a guide, the lateral width 143 refers to the lateral width of such a force-mediating part.

In some embodiments, both the first 170 and second 172 force direction transferring connections has a respective radius of curvature 171, 173 of at least 2.5 cm, or even at least 5.0 cm, for a medium sized protective garment 100 according to EN 13402. This minimum radius of curvature is preferably valid throughout the whole force direction transferring connection 170, 172 in question, such as in the example illustrated in FIG. 1c along the entire curved part interconnecting the first element part 140 to the second 150 or third 160 element part.

It is noted that the force direction transferring connections 170, 172 can be arranged in the form of an edge or demarcation line between different fields of the protective garment 100, such as is exemplified in FIG. 1c. It is noted, however, that the force direction transferring connections 170, 172 can also be provided in other ways, such as using tension strings (see below). The radius of curvature 171, 173 refers to a local radius of curvature of a structure active to distribute the forces F1, F2, F3 between the element parts 140, 150, 160 while altering the direction in which the force F1, F2, F3 in question acts.

Even though smoothly curved force direction transferring connections 170, 172 are sometimes preferred for aesthetical reasons, it is also foreseeable to have only part-wise continuous, or even part-wise straight, force direction transferring connections 170, 172. For instance, each force direction transferring connection 170, 172 could have a polygon shape approximating a curved shape along which the forces F1, F2, F3 are distributed. In case a force direction transferring connection 170, 172 is provided as a tension string, the tension string can correspondingly change direction abruptly as it passes a reinforcement part (see below). In such cases, the radius of curvature 171, 173 described herein can correspondingly pertain to a radius of curvature of a corresponding curved shape approximated by such a part-wise continuous force direction transferring connection 170, 172.

Another way to look at this is that, for each force direction transferring connection 170, 172, the tension force F1 along the intergluteal cleft of the standing user wearing the protective garment is distributed to and from the respective tension force F2 and F3, along and by a path defined by the force direction transferring connection 170, 172 in question, the path comprising one or more, such as at least two, bends between the first element part 140 and the second/third element part 150, 160 in question.

As is further illustrated in FIGS. 1a and 1b, the back part 101 can comprise a respective buttock part 102b, being laterally separated and each arranged to cover a buttock of the standing user wearing the protective garment 100. Each such buttock part 102b can comprise a convex bowl shape in a relaxed state of the protective garment 100, the bowl shape being arranged to at least partly follow a corresponding shape of the buttock of the standing user wearing the protective garment 100. This achieves a gripping effect, maintaining the protective garment 100 in place even when the first tension force F1 pulls the back part 101 forward and when the user moves around. It has turned out that the buttocks part 102b also prevents excess textile material of the protective garment 100 from moving into the intergluteal cleft of the wearing user during use, which typically is not comfortable.

The buttock part 102b can be provided as a local surplus of fabric, such as in any of the applicable ways exemplified above to affect the properties of the fabric (such as using seamless knitting or application of various materials onto or into the fabric so that it bulges in a relaxed state, or using molding of the material so as to achieve a locally non-flat shape of the material). The buttock part 102b can also be provided as a locally different material, being more elastic as compared to a surrounding material, locally provided in the area of the buttock part 102b. The buttock part 102b can also be provided with a friction-increasing layer arranged to abut directly against the wearing user's skin, such as a silicone flock or other friction-increasing layer. In case the dorsal shaping element 130 is partly or fully provided as a separate piece of fabric from the back part 102, the buttock part 102b can be provided as an integrated part of the back part 102, forming a distinct part in relation to the dorsal shaping element 130. In case the protective garment 100 comprises an outer layer, such as a layer arranged outwardly of the back part 102, the buttock part 102b can also be provided as a part of such outer layer. In general, the buttock part 102b can be provided as a part of an outer shell layer of the protective garment 100 in case such an outer shell layer is provided.

As is also illustrated in FIGS. 1a and 1b, the protective garment 100 can further comprise a dorsal line 180. Such a dorsal line 180 can be formed from a fabric forming part of the protective garment 100, such as being achieved using a seam locally increasing the firmness and/or decreasing the elasticity along the dorsal line 180, or being achieved by the fabric being locally folded (the fold being fixated using a seam, a weld, a glue or in any other suitable manner), such as inwards towards the skin of the standing user wearing the protective garment 100.

The dorsal line 180 can hence be arranged so that the fabric locally protrudes inwards, towards the user, along the intergluteal cleft of the user. Moreover, the dorsal line 180 can run at least along, and overlapping with, part of the first element part 140, or along the entire first element part 140. The dorsal line 180 can also connect to the waist 103. Hence, the dorsal line 180 can be formed in the material forming the dorsal shaping element 130, and in particular in the material forming first element part 140. The dorsal line 180 can also connect to the leak-proof body 120, such as at the perineum of the standing user wearing the protective garment 100.

The dorsal line 180 can run in its entirety in the median plane of the standing user wearing the protective garment 100.

In some embodiments, the dorsal line 180 has a curved shape, in a relaxed state of the protective garment 100, the curved shape being arranged to follow a shape of the intergluteal cleft of the standing user wearing the protective garment 100. This is illustrated in FIG. 1a. The curved shape can be achieved by the material of the dorsal line 180 being folded and properly fixated into a curved fold. In some embodiments, the fold can gradually or abruptly end some distance, such as at least 5 cm, from a dorsal end of the dorsal line 180, even if the dorsal line 180 itself (such as in the form of a seam) can continue upwards, even all the way to the waist 103. For instance, the width of a folded piece of fabric can decrease to zero at the distance from the dorsal end of the dorsal line 180.

It has turned out that such a dorsal line 180 aids in the first element part 140 being maintained in the intergluteal cleft of the user during use, by maintaining the first element part 140 in the intergluteal cleft of the wearing user even when the user moves around. The dorsal line 180 is then active in the lateral control of the first element part 140.

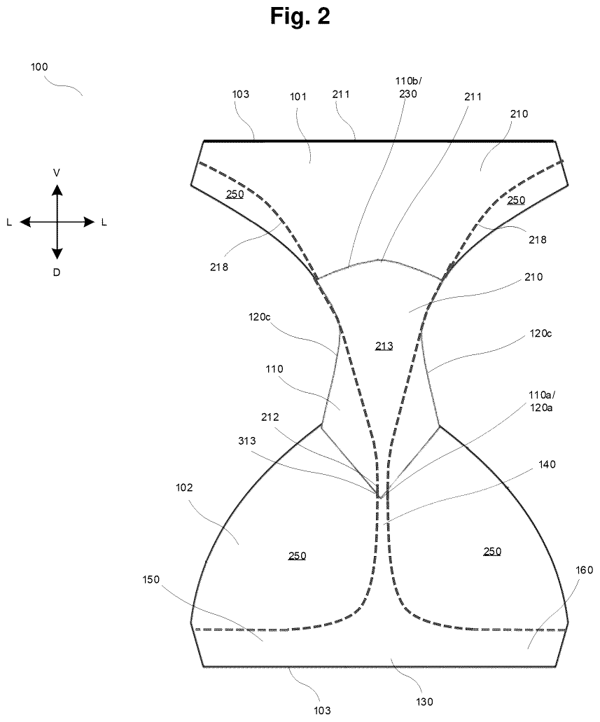

FIG. 2 illustrates another embodiment of a protective garment 100, in which the dorsal shaping element 130 extends from the crotch part 110 backwards/upwards to the hips of the standing user wearing the protective garment 100 and ventrally forward to the belly of the user. Ventrally of a point where the dorsal shaping element is fastened to the crotch part 110, the protective garment 100 further comprises a ventral shaping element 210, extending from a point where the shaping element 210 is fastened to the crotch part 110, ventrally up along the lower belly of the standing user wearing the protective garment 100.

As illustrated in FIG. 2, the second 150 and third 160 element parts of the dorsal shaping element 130 can connect to the ventral shaping element 210 so that the dorsal shaping element 130 and the ventral shaping element 210 together form a thong-like structure, or together form at least the bottom parts of a thong-like structure. The dorsal shaping element 130 and the ventral shaping element 210 can be connected to each other along the crotch part 110, or the crotch part 110 can constitute a connection between the dorsal shaping element 130 and the ventral shaping element 210.

Side edges 218 of the ventral shaping element 210 can diverge ventrally to connect to the dorsal shaping element 130 at or near the waist 103. As discussed above, the dorsal shaping element 130 can extend all the way up to the waist 103 or end beneath the waist 103; and the corresponding can be true for the ventral shaping element 210.

Hence, the dorsal shaping element 130 and the ventral shaping element 210 can form, possibly together with the crotch part 110, a thong-like structure including a thong waist band. This thong-like structure can be provided as material modifications and/or additions to the material of the rest of the protective garment 100, or be provided, in part or entirely, as one or more separate layers of fabric in addition to one or more layers of fabric constituting the rest of the protective garment. The latter case will be exemplified in detail below.

The thong-like structure is at least fastened to the rest of the protective garment 100 by it being fastened to the crotch part 110 as described above, and in particular to the leak-proof body 120.

In one particular example, the protective garment 100 comprises the thong-like structure, in turn comprising the dorsal shaping element 130 and the ventral shaping element 210, and the protective garment 100 in addition comprises one or several wing parts 250 attached ventrally along sides of the thong-like structure so as to form a protective garment 100 arranged to cover more of the body of the user wearing the protective garment 100. It is understood that such wing parts 250 can form various parts of different types of garments, such as leg parts of trousers or shorts.

In this and in other embodiments, the outer lateral contours of the thong-like structure can comprise elastic elements arranged to be elongated, against a longitudinal spring force of the elastic elements, as a result of the user puts the protective garment 100 on. Such elastic elements can be provided on or between any layer comprised or not comprised in the thong-like structure itself.

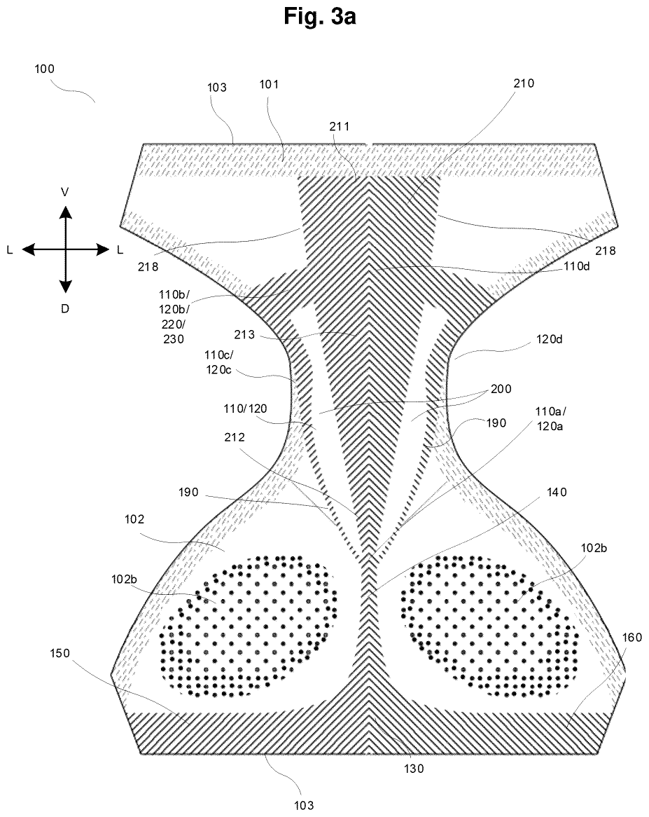

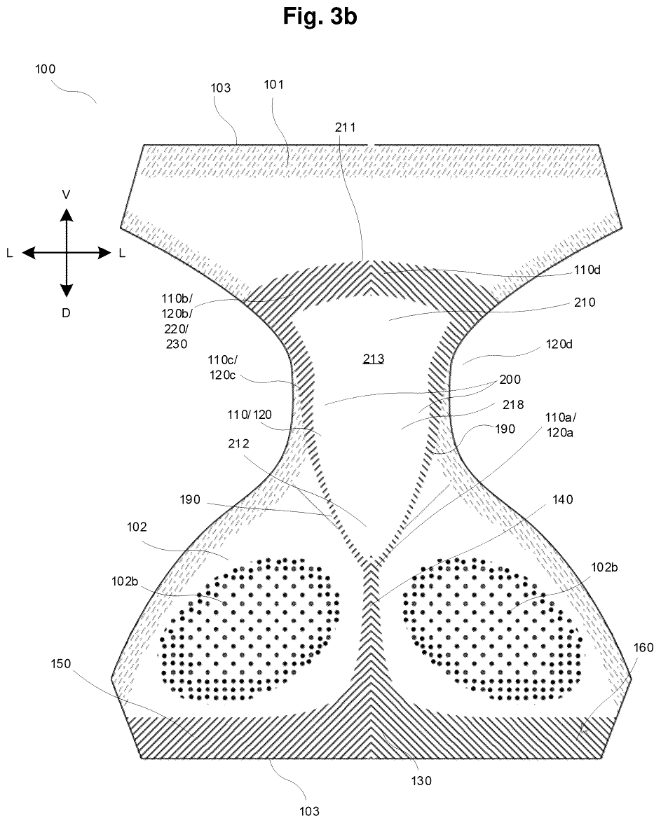

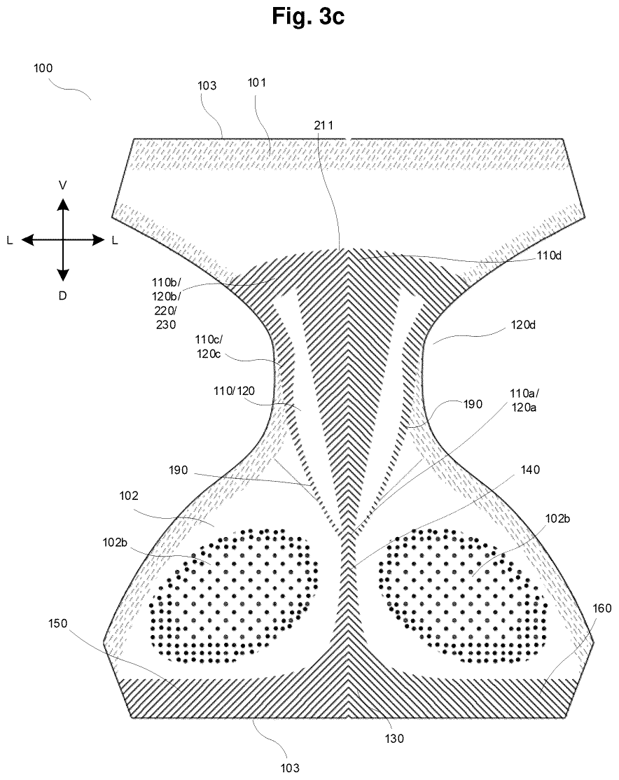

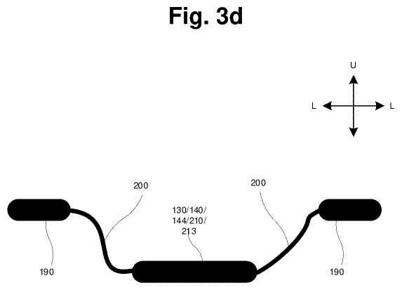

FIGS. 3a, 3b and 3c illustrate three different embodiments of a protective garment 100 that can be manufactured by selectively varying local properties of the fabric of the protective garment 100, such as using seamless knitting, the application of silicon flocking, and so forth. However, it is understood that the structures schematically illustrated in FIGS. 3a-3c can also be provided using different material layers with different material properties or by any other suitable technique, as has been exemplified and discussed above.

In the examples shown in FIGS. 3a-3c, but also generally, the waist 103 can be associated with a lateral tension force, along the waist 103 when the protective garment 100 is worn by the user, the lateral tension force being equal to or firmer as compared to a corresponding lateral tension force arising during wearing of the protective garment 100 in the front part 101/back part 102 fabric directly beneath the waist 103. This way, the protective garment 100 can more easily be held up as the wearing user moves about. In other embodiments, the waist 103 has a less firm tension force (with the corresponding meaning) as compared to the front part 101/back part 102 fabric directly beneath the waist 103. Generally, the waist 103 can have the same firmness and the same vertical height at the front of the protective garment 100, when worn by the standing user, as at its back.

Moreover, side edges 110c of the crotch part 110, as well as any side edges of the front part 101 and/or the back part 102 forming edges of leg openings, can also be provided with firm edge material, such as of the general type described in relation to the waist 103. This will prevent the back part 102 to “hike up” when the protective garment 100 is worn and the wearing user moves about.

As is illustrated in FIGS. 3a, 3b and 3c, the protective garment 100 can comprise a pair of laterally separated shaping elements 190, each running generally ventrally from the first element part 140, in relation to the standing user wearing the protective garment 100, along either side of the crotch part 110, such as along either side of the leak-proof body 120, to a position ventrally of the perineum of the standing user.

The laterally separated shaping elements 190 can be arranged to apply a tension force in the ventral-dorsal direction when the protective garment 100 is worn by the standing user. This can, for instance, be achieved by the laterally separated shaping element 190 being manufactured, for instance in any of the ways discussed above, with elastic properties, pulling together the material of the crotch part 110 when the protective garment 100 is in a relaxed state and the material of the crotch part 110 being extended as a result of the user pulling the waist 103 up, thereby stretching the laterally separated shaping elements 190.

The laterally separated shaping elements 190 can be arranged to extend on either lateral side of the labia of a standing female user wearing the protective garment 100. Moreover, each of the laterally separated shaping elements 190 can be arranged to extend ventrally from the first element part 140, for instance from a connection point between the first element part 140 and the leak-proof body 120, and also be arranged so that, when a ventral/upward force is applied to the front part 101 and the back part 101 as a result of the waist 103 being pulled upwards in relation to the user wearing the protective garment 100, the laterally separated shaping elements 190 are in turn forced upwards.

Such forcing upwards can then also result in that the leak-proof body 120 is urged upwards towards or even into direct contact with the labia (or into indirect contact in case of any additional layers of material between the leak-proof body 120 and the wearing user's skin).

This urging upwards can be achieved by the laterally separated shaping elements 190 being fastened along their entire length, or at one or several distinct fastening points along their length, to the crotch part 110 and/or directly to the leak-proof body 120 (see below and FIG. 3d).

As is illustrated in FIGS. 3a-3c, the dorsal shaping element 130, such as the first element part 140, can be directly connected to each of the laterally separated shaping elements 190. This can be achieved in different ways, such as the first element part 140 and the laterally separated shaping elements 190 being formed as one integrated shape or body, being manufactured in the same manner; or via a fastening, such as a stitch, weld or bond, at the ventral end 141 of the first element part 140.

As is also illustrated in FIGS. 3a-3c, the dorsal shaping element 130, together with the laterally separated shaping elements 190, can form a “Y”-shape. Such “Y”-shape can then be arranged to run peripherally and at least partly enclose the labia of the standing female user wearing the protective garment 100.

The laterally separated shaping elements 190 can be arranged to run, generally and/or at least along one or many subsections, in parallel to the first element part 140, along the ventral-dorsal direction of the wearing user. It is noted that the laterally separated shaping elements 190 in other embodiments can be arranged to run, generally and/or at least along one or many subsections, in parallel to a part of the ventral shaping element 210, and in particular in parallel to a section 213 of the ventral shaping element 210 that runs across the leak-proof body 120.

In some embodiments, a lateral width 143 of one or more sections 144 of the first element part 140 running across the leak-proof body 120 and in parallel, in said manner, to the laterally separated shaping elements 190, is between 30 mm and 60 mm for a medium sized protective garment 100 according to EN 13402.

Correspondingly, a lateral width 143 of one or more sections 213 of the ventral element part 210 running across the leak-proof body 120 and in parallel, in said manner, to the laterally separated shaping elements 190, can be between 30 mm and 60 mm for a medium sized protective garment 100 according to EN 13402.

In both cases, the lateral width can be measured at the gracilis of the standing user wearing the protective garment 100.

As is illustrated in FIG. 3d, a respective side part 200 of the protective garment 100 located, on the one hand, between the section 144 of the first element part 140 or the section 213 of the ventral shaping element 210 and, on the other hand, either of the laterally separated shaping elements 190, can be made from a material which is less firm and/or more resilient than that of the section 144/213 in question and less firm and/or more resilient than that of the laterally separated shaping element 190 in question. This allows the laterally separated shaping elements 190 to press up against the skin of the standing female user wearing the protective garment 100 on either side of her labia, whereas the dorsal 130 or ventral 210 shaping element together with the side parts 200 can cup around the labia, for a snug and secure fit as the user moves around.

The laterally separated shaping elements 190 can be arranged to follow side edges 110c of the crotch part 110 and/or side edges 120c of the leak-proof body 120.

As is illustrated in FIGS. 3a-3c, but also in FIGS. 1a, 1b, 1d and 2, a ventral end 211 part of the ventral shaping element 210 can be arranged to extend laterally across the lower belly of the standing user wearing the protective garment 100. This ventral end 211 part can then be arranged to cause a tension force to arise laterally along the ventral end 211 part as a result of the standing user wearing the protective garment 100, for instance by the ventral end 211 part being less elastic than materials of the protective garment 100 surrounding the ventral end 211 part of the shaping element 210.

As is illustrated in FIGS. 2, 3a, 3b and 3c, the ventral shaping element 210 can extend ventrally from the crotch part 110, such as from the leak-proof body 120, to the waist 103. In a way corresponding to the dorsal shaping element 130, the ventral shaping element 210 can be fastened to the crotch part 110 or to the leak-proof body 120 at a dorsal end 110a, 120a thereof; at a ventral end 110b, 120b thereof; and/or at any other location along a dorsal-ventral extension of the crotch part 110 or the leak-proof body 120.

The dorsal shaping element 130, such as the first element part 140, and the ventral shaping element 210 can be interconnected at a ventral end 141 of the dorsal shaping element and a dorsal end 212 of the ventral shaping element 210, or the dorsal shaping element 130 can be separated from the ventral shaping element 210 by the crotch part 110 or leak-proof body 120. Irrespectively, in case a ventral shaping element 210 is used, the dorsal shaping element 130, the ventral shaping element 210 and possibly also the crotch part 110 or leak-proof body 120 can be fastened together so as to form the thong-like structure. This thong-like structure can then be fastened to the crotch part 110, for instance to the leak-proof part 120, at least at one point along the ventral-dorsal length of the crotch part 110 or leak-proof body 120.

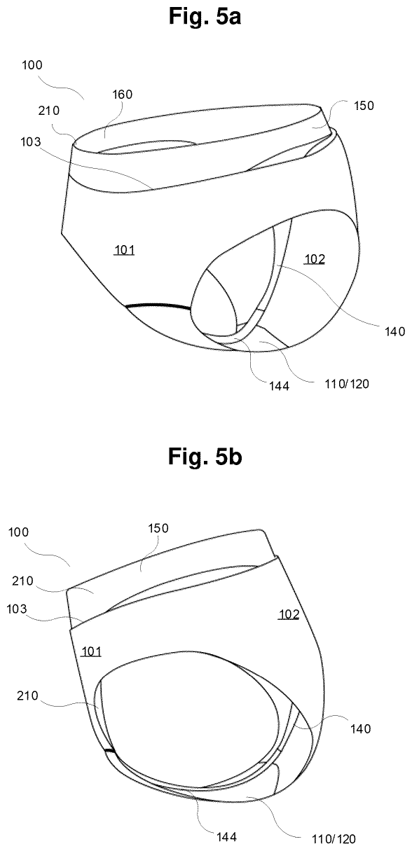

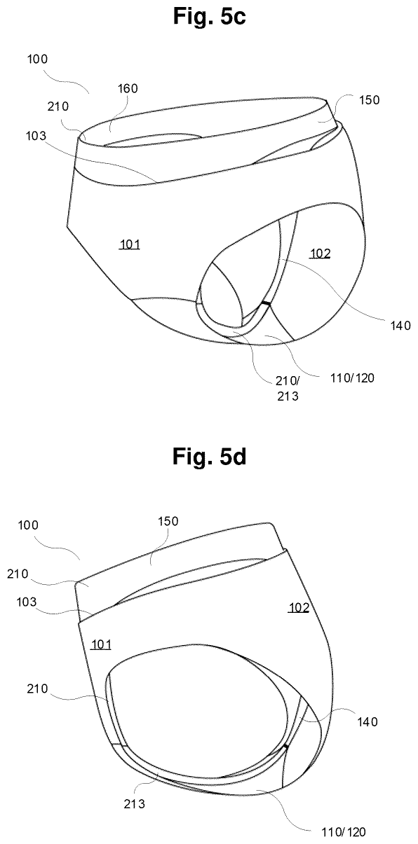

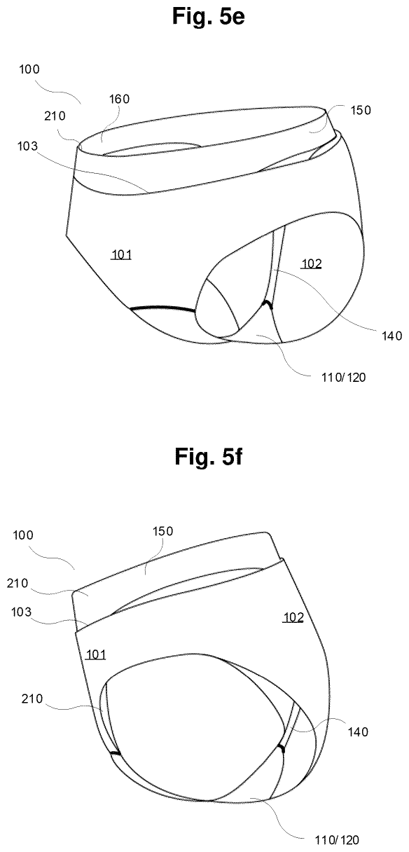

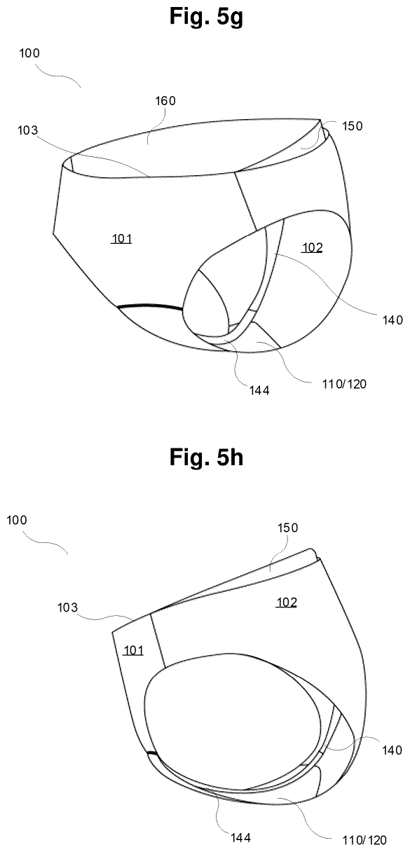

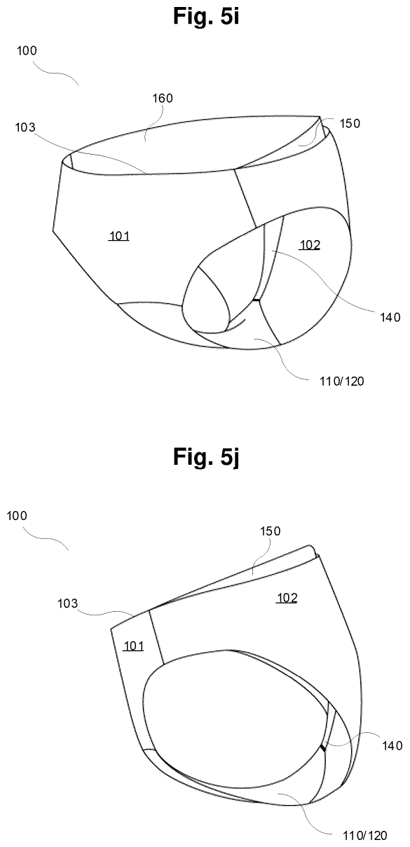

As illustrated in FIGS. 3a and 3b, the ventral shaping element 210 can be tapered so that a ventral end 211 thereof is wider than a dorsal end 212 thereof. The ventral end 211 can run or not run up to the waist 103. In some embodiments, the dorsal end 212 is the same as, or overlaps with a friction-increasing layer 220 and/or a ventral gripping element 230 as will now be described. In some embodiments, the ventral element 210 can be curved upwards-outwards (laterally outwards). In some cases, this curving can even be so that a ventrally running first ventral element part extends up to, and connects to a pair of laterally running second and third ventral element parts, via a force-mediating direction-changing element in a way that would generally correspond to the first element part 140, the second element part 150, the third element part 160, the first curved force direction transferring connection 170 and the second 172 curved force direction transferring connection of the dorsal element part 130. See, for instance, FIGS. 5a and 5b for an example of this.

Namely, the protective garment 100 can comprises a friction-increasing layer 220 arranged to abut directly against the skin of the standing user wearing the protective garment 100, the friction-increasing layer 220 being provided to abut directly against the skin at the lower belly of the user. The friction-increasing layer 220 can be provided as a silicon flock on an innermost surface layer facing inwards on the protective garment 100, such as provided on or as a part of the ventral shaping element 210, or be in the form of an applied “surplus penetration” as described above. It can have a shape that gently bulges ventrally upwards with respect to the standing user wearing the protective garment 100. Other surface friction-increasing techniques can also be used, such as providing locally applied silicon or rubber threads across the friction-increasing layer 220.

The protective garment 100 can also comprise a ventral gripping element 230, that may also form a part of the ventral shaping element 210. The ventral gripping element 230 can be generally laterally elongated and can have a convex shape softly bulging ventrally/upwards in relation to the standing user wearing the protective garment 100. In case the protective garment 100 comprises both a friction-increasing layer 220 and a gripping element 230, these two may overlap partially or completely.

The ventral gripping element 230 can be less elastic and/or firmer and/or less flexible, in/along its lateral direction, than materials of the protective garment 100 surrounding the ventral gripping element 230. The ventral gripping element 230 can also be arranged to connect on each lateral side to a side edge 110c of the crotch part 110, and can be arranged along or ventrally of a ventral end edge 120b of the leak-proof body 120.

Such a ventral gripping element 230 can be arranged to achieve a gripping effect around the mons pubis for a female user wearing the protective garment 100, effectively preventing the protective garment 100 from sliding dorsally due to the gripping effect. This in turn achieves that the crotch part 110 is held in position to achieve a secure fit of the protective garment 100.

As is illustrated in FIG. 3b, the ventral shaping element 210 can also be arranged not to run across the crotch part 110 or the leak-proof body 120, in which case the protective garment 100 can anyway comprise the friction-increasing layer 220 and/or the ventral gripping element 230.

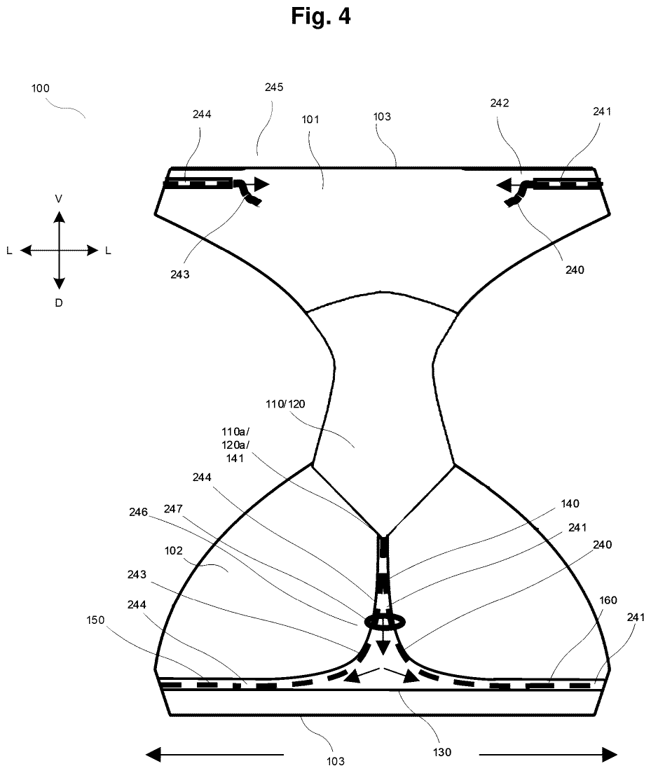

FIG. 4 illustrates an embodiment in which the dorsal shaping element 130 is or comprises a mechanism using which the above-discussed tension forces F1, F2, F3 can be adjusted manually by a user wearing the protective garment 100. This is accomplished by two pull strings 240, 243 arranged to run along, or constitute, the first 140, second 150 and third 160 element parts. In alternative embodiments, such pull strings 240, 243 are arranged to run along, or constitute, the first 140, second 150 and third 160 element parts but are not adjustable. In the latter case, the pull strings 240, 243 can be arranged to achieve the tension forces F1, F2, F3 by their length between fastening points to the rest of the protective garment and/or their selected elastic properties.

It is understood that the pull strings 240, 243 can be elastic or inelastic, and arranged to achieve the tension forces F1, F2, F3 alone or in combination with any type of first 140, second 150 or third 160 element parts of the types described above.

Generally, the protective garment 100 can comprise a first pull string 240, running along the first element part 140 and the second element part 150. The protective garment 100 can also comprise a second pull string 243, running along the first element part 140 and the third element part 160. It is noted that both pull strings 240, 243 then run along or constitute the first element part 140; the first pull string 240 runs along or constitutes the second element part 150; and the second pull string 243 runs along or constitutes the third element part 160.

Furthermore, the first 240 and second 243 pull strings can run in respective channels 241, 244 or along corresponding respective guides, the channels or guides following a shape of the element parts 140, 150, 160 in question. The channels 241, 244 can, for instance, be sewn channels or channels formed otherwise (such as using gluing or welding, or as a stitched tunnel) in the fabric forming the element part 140, 150, 160 in question, or correspondingly formed between fabric layers. Exit and/or entry holes for the pull strings 240, 243 can be formed on an inside and/or an outside of the protective garment 100. Corresponding guides can be, for instance, in the form of a series of textile, plastic or metal eyes, hooks or holders.

Each of the first 240 and second 243 pull strings can be arranged to be tightened so as to increase the first and/or second tension force by decreasing the length of the pull string 240, 243 in question, such as using a first tightening means 242 and a second tightening means 245.

In some embodiment, such as the one illustrated in FIG. 4, the first 240 and/or second 243 pull string can be fastened, or arranged to be fastened, such as via the tightening means 242, 245 in question, to the protective garment 100 on the front part 101, at a point ventrally to the iliac crest of the standing user wearing the protective garment 100. This corresponds to what has been described above, regarding the second 150 and third 160 element parts running across and around the hip of the wearing user to terminate on the front side of the user, such as at the wearing user's belly.

At the opposite end of the first 240 and/or second 243 pull string, the pull string 240, 243 in question can be directly fastened to, or be arranged to be directly fastened to, the leak-proof body 120; or indirectly fastened to, or arranged to be indirectly fastened to, the leak-proof body 120 via a connecting part that is less flexible than material laterally surrounding the connecting part. For instance, such a connecting part can be a plastic, metal or inelastic fabric part connecting the pull string 240, 243 to the leak-proof body 120. The connecting part can also be a variable tightening means of a type as described herein in relation to tightening means 242, 245.

In general, the protective garment 100 can comprise one or two variable tightening means 242, 245 arranged to tighten one or both of the pull strings 240, 243 at the hip of the wearing user or front part 101 of the protective garment 100 and/or one or two corresponding tightening means somewhere else along the first 140, second 150 and/or third 160 element parts, such as at a connection of the pull string 240, 243 in question to the leak-proof body 120.

The tightening means 242, 245 can comprise a drawstring construction, a chord lock, a press button or button/buttonhole combination, a quick-release fastener, or any other suitable device arranged to tighten and hold the pull string 240, 243. For instance, the pull string 240, 243, or each pull string 240, 243, can have a series of through holes or push buttons arranged to engage with a corresponding button or push button provided at the front part 101. In other examples, the pull string 240, 243, or each pull string 240, 243, can be arranged with a swan hook, arranged to engage with and fasten to ladder straps at the front part 101. The tightening can be along a continuous scale or according to a series of discrete settings. Preferably, the tightening means 242, 252 has a generally flat construction. It can comprise at least one part made from a rigid material, such as plastic or metal.

As is also illustrated in FIG. 4, the first 170 and/or second 172 curved force direction transferring connection can be reinforced by a reinforcing element 246 engaging with the first 240 and/or second 243 pull string and forcing the pull strings 240, 243 in question in a direction laterally towards each other, or holding the pull strings 240, 243 at a minimum lateral distance from each other at the reinforcing element 246. To do this, the reinforcing element 246 can comprise a rigid element 247, such as a closed rigid structure through which one or both of the pull strings 240, 243 run, such as a plastic or metal ring. The reinforcing element 246 can also comprise a reinforced fabric or textile material part, for instance provided with an extra layer of fabric, an extra stiff or sturdy layer of fabric, an extra seam and similar, offering a locally arranged reinforcement in turn pulling the pull string 240, 243 laterally. The reinforcing element 246 can be arranged to engage with both the first 240 and the second 243 pull string, and thereby to keep them at a minimum lateral distance from each other at a point near the dorsal end 142 of the first element part 140. Alternatively, one or several separate reinforcing elements can be used for one or each of the pull strings 240, 243.

Using the pull strings 240, 243, the user wearing the protective garment 100 can manually adjust the tension forces F1, F2, F3 so as to achieve a comfortable, secure and desired snug fit of the protective garment 100, and in particular of the dorsal element 130 (thong-like structure), depending on personal taste, body type, activity to perform and so forth.

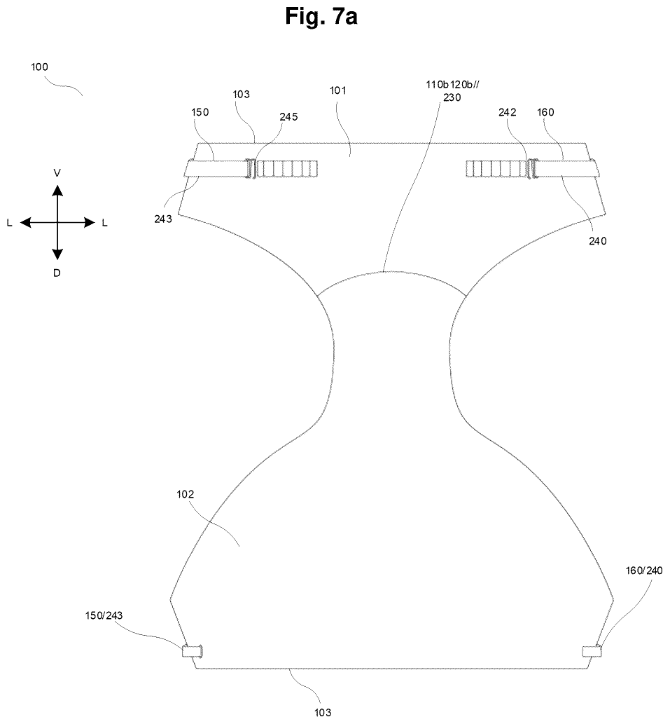

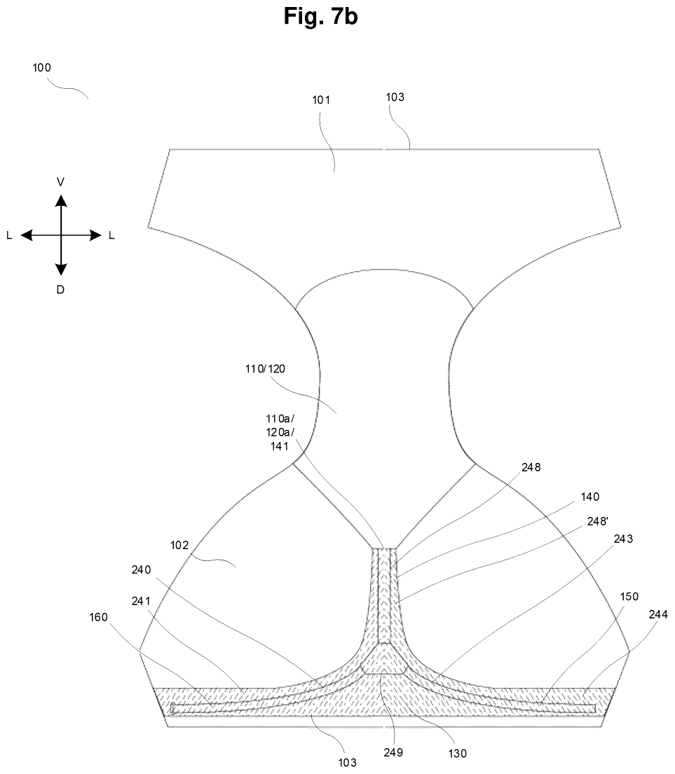

FIGS. 7a and 7b show an alternative embodiment using pull strings 240, 243.

In contrast to the embodiment illustrated in FIG. 4, in FIGS. 7a and 7b the first pull string 240 and the second pull string 243 do not each run along the first element part 140. Instead, the first 240 and second 243 pull strings are attached to a joining means 249, such as a piece of fabric or rigid element, such as a piece of plastic or metal. The joining means 249 can be relatively rigid and/or relatively inelastic in relation to fabric surrounding the joining means 249, and in particular in relation to flexible material forming a dorsal channel 248′ in and along which the joining means 249 can be arranged to be movable in a dorsal-ventral (upwards-downwards) direction along the general body shape of the wearing user.

More concretely, in this embodiment the first pull string 240 and the second pull string 243 are both interconnected to a dorsal pull string 248 via the joining member 249.

The dorsal pull string 248 runs along the first element part 140, and can even constitute the first element part 140.

The joining member 249, however, is arranged to run in and along the dorsal channel 248′. The dorsal channel 248′ can be tapered so that the joining member 249 engages with an inside of the dorsal channel 248′ if moved ventrally in the dorsal channel 248′ so that the joining member 249 is prevented from moving ventrally in the dorsal channel 248′ beyond a certain point, such as at least 5 cm dorsally of the leak-proof body 120. The dorsal pull string 248 can also run in the dorsal channel 248′.

The joining member 249 can be generally triangular, fastening to each of the first 240, second 243 and dorsal 248 pull string in a respective corner of the joining member 249.

It is realized that the joining member 249 can constitute or form part of the first 170 and/or second 172 force direction transferring connection, by the joining member 249 having a curved shape between the triangle corners. However, other or additional first 170 and/or second 172 force direction transferring connection can also be used, such as the types of mechanisms described above in connection to FIG. 4.

The joining member 249 and the dorsal pull string 248 provide for an even lateral and dorsal/ventral distribution of tension force, even as the wearing user moves around, providing an excellent secure and snug fit of the protective garment.

As the waist 103 and/or back part 102 is pulled up, the joining member will be forced ventrally in the dorsal channel 248′ by the pulling force from the dorsal pull string 248, until the joining member 248 engages with the dorsal channel 248′. Then, the joining member 248 will pull the first 240 and second 243 pull strings so as to achieve the above-described mediation/distribution of tension forces F1, F2, F3.

In some embodiments, the dorsal pull string 248 is less elastic than the first 240 and second 243 pull strings, in respective longitudinal direction. It may even be so that the dorsal pull string 248 is inelastic, whereas the first 240 and second 243 pull strings are elastic.

As is seen in FIGS. 7a-7b, the first 240 and second 243 pull strings run in channels 241, 244 and exit channels 241, 244 via through holes on the front part 101 so as to allow tightening. This way, the entire dorsal shaping element 130 construction, apart from the tightening mechanism, can remain hidden inside the protective garment 100.

The embodiments of FIGS. 4 and 7a-7b can be combined in various ways. For instance, the independently moving first 240 and second 243 pull strings in FIG. 4, from leak-proof body 120 to tightening means 242, 245, can provide reliable fit as the user moves vigorously or with large leg movements, as the second F2 and third F3 tension forces can be mediated more independently. In FIGS. 7a and 7b, the dorsal pull string 248 can be arranged as two separate pull strings, each being connected to a respective one of the first 240 and second 243 pull strings at the joining member 249, such as laterally opposite corners of its triangular shape. This will provide a similar independence between the second F2 and third F3 tension forces. In other embodiments, the joining member 249 can be flexible and/or elastic, and/or otherwise not arranged to be movement-limited in the dorsal direction by the dorsal channel 248′, constituting the curved direction transferring connection 170, 172 to distribute the forces F1, F2, F3. In the latter embodiments, the dorsal pull string 248 can be resilient.

FIGS. 5a-5l illustrate six different embodiments of a protective garment 100: FIGS. 5a and 5b show a first such embodiment from different perspectives; FIGS. 5c and 5d illustrate correspondingly a second such embodiment; and FIGS. 5e, 5f; 5g, 5h; 5i, 5j; and 5k, 5l illustrate third, fourth, fifth and sixth such embodiments.

In all embodiments shown in FIGS. 5a-5l, the dorsal shaping element, for instance particularly the first element part 140, is arranged at least partly as a separate, overlapping, flexible layer in relation to the rest of the protective garment 100. In these and other embodiments, the dorsal shaping element can be non-connected to, i.e. freely movable in relation to, the protective garment 100 at least along a stretch of the dorsal shaping element running between a first fastening point, to the leak-proof body 120, and a second fastening point, to the rest of the protective garment 100. The second fastening point can be arranged at least 5 cm, or at least 8 cm, or at least 10 cm, for a medium sized protective garment (100) according to EN 13402, from the first fastening point and/or laterally at least at or in front of the iliac crest of the standing user wearing the protective garment and/or vertically at the waist 103.

In these and other embodiments, the dorsal shaping element 130 can be fastened to the leak-proof body 120, such as sewn to the leak-proof body 120.

In these and other embodiments, the dorsal shaping element 130 can run internally to the leak-proof body 120 when the protective garment 100 is worn by the standing user.

That the dorsal shaping element 130 runs internally to the leak-proof body 120 means that the dorsal shaping element 130, and in particular the section 144 (the section running across the leak-proof body 120), runs between the leak-proof body 120 and the skin of the user wearing the protective garment 100, across the leak-proof body 120.

Correspondingly, the ventral shaping element 210 can comprise the section 213 running across the leak-proof body 120, in which case the section 213 can run internally to the leak-proof body 120 in relation to the wearing user. Also, the ventral shaping element 210 can be fastened (ventrally or dorsally) to the leak-proof body 120 in a manner corresponding to what has been the in relation to the fastening to the leak-proof body 120 of the dorsal shaping element 130.

In some embodiments, the dorsal shaping element 130 is fastened to the protective garment 100 at the waist 103.

In such and other embodiments, the second element part 150 and the third element part 160 can both be fastened to the protective garment 100 at a respective point ventrally to the iliac crest of the standing user wearing the protective garment 100, so that each of the second element part 150 and the third element part 160 runs, from the dorsal end 142 of the first element part 140, around the iliac crest of the standing user wearing the protective garment 100 and again laterally towards but not up to a thought front center line of the front part 101.

Hence, the dorsal shaping element 130 and/or the ventral shaping element 210 can be in the form of separate layers of textile that are only fastened to the rest of the protective garment 100 at one or several discrete points. In some embodiments, such a fastening point is at least arranged at the leak-proof body 120. In FIGS. 5a-5l, fastening points between the dorsal shaping element 130 and/or the ventral shaping element 210 are marked using fat lines.

As described above, the dorsal shaping element 130 can be specifically fastened to the dorsal end 120a of the leak-proof body 120 and/or to the ventral end 120b of the leak-proof body 120. Similarly, the ventral shaping element 210 can be specifically fastened to the dorsal end 120a of the leak-proof body 120 and/or to the ventral end 120b of the leak-proof body 120. These fastening types can be varied, but it is preferred that the dorsal shaping element 130 does not overlap with the ventral shaping element 210 in the ventral-dorsal direction and in the vicinity of the leak-proof body 120.

FIGS. 5a and 5b illustrate an embodiment in which the dorsal shaping element 130 is fastened to the ventral end 120b of the leak-proof body 120, and runs over the leak-proof body 120 internally to the leak-proof body 120 between the dorsal end 120a and the ventral end 120b. It is noted that, in FIGS. 5a and 5b, the dorsal shaping element 130 and the ventral shaping element 210 are interconnected near the waist 103 so as to form a thong-like structure which is only joined to the rest of the protective garment 100 at the ventral end 120b of the leak-proof body 120.

In these and other embodiments, a section 144 of the dorsal shaping element 130 (or correspondingly a section 213 of the ventral shaping element 210, see FIGS. 5c and 5d) that runs across the leak-proof body 120 is tapered, so that a dorsal end of the section 144, 210 is narrower than a ventral end of the section 144, 210, in relation to the standing user wearing the protective garment 100.

Also, in these and other embodiments, the section 144, 214 in question can be laterally narrower than the leak-proof body 120.

FIGS. 5c and 5d illustrate an embodiment in which the ventral shaping element 210 is instead fastened to the leak-proof body 120 at its dorsal end 120a and runs free from the leak-proof body 120 from this point ventrally.

In this and other embodiments, the ventral shaping element 210 can be fastened to the protective garment 100 at the waist 103 thereof, and/or be connected to the dorsal shaping element 130, such as connect to the second 150 and third 160 element parts as illustrated in FIGS. 5c and 5d to form the thong-like structure. At any rate, the ventral shaping element 210 can then be non-connected to the protective garment 100 between such fastening points (at the waist 103 and/or to the dorsal shaping element 130 on the one hand, and on the other hand to the leak-proof body 120). An upper connection between the ventral shaping element 210 and the dorsal shaping element can be near the waist 103.

FIGS. 5e and 5f illustrate an embodiment where the ventral shaping element 210 is fastened to the ventral end 120b of the leak-proof body 120 and the dorsal shaping element 130 is fastened to the dorsal end 120a of the leak-proof body 120, the leak-proof body 120 forming a lower connection between the ventral shaping element 210 and the dorsal shaping element 130.

In case the ventral shaping element 210 and/or the dorsal shaping element 130 (such as the first element part 140) is fastened to the leak-proof body 120, such fastening can be at, or at least overlapping with, the perineum of the standing user wearing the protective garment 100.

In all embodiments illustrated in FIGS. 5a-5f, the dorsal shaping element 130 and the ventral shaping element 210, and possibly the leak-proof body 120, together form the thong-like structure.

The embodiment shown in FIGS. 5g and 5h, however, is a variant in which a separate-layer dorsal shaping element 130 is fastened to the ventral edge 120b of the leak-proof body 120 and connects, via the second 150 and third 160 element parts, to the front part 101 of the protective garment 100 after having passed the iliac crest of the wearing user on either side, all the time running internally to the back side of the protective garment 100. The ventral shaping element 210, if used at all in this example, is not in the form of a freely movable layer of material.

The embodiment illustrated in FIGS. 5i and 5j is similar to the one shown in FIGS. 5g and 5h, but here the dorsal shaping element 130 is instead fastened to the dorsal end 120a of the leak-proof body 120.

Finally, in the embodiment illustrated in FIGS. 5k and 5l, the dorsal shaping element 130 is not in the form of a freely movable layer of material, but the ventral shaping element 210 is. The ventral shaping element 210 connects to the ventral end 120b of the leak-proof body 120 and then connects to the dorsal shaping element 130 at the back part 102 of the protective garment 100.

It is realized that the different embodiments illustrated in FIGS. 5a-5l are mere examples of the different ways the present principles can be combined, and that there are numerous additional possibilities to apply the principles in different combinations.

In these and in other embodiments, the first element part 140, and in particular a section of the first element part 140 arranged to be inside the intergluteal cleft of the standing user wearing the protective garment 100, is made from a material that can breathe, in other words a material that is permeable to water vapour. In particular, such breathable material can be more permeable to water vapour as compared to other parts of the dorsal shaping element 130. For instance, the first element part 140 or the section can comprise perforations or loosely weaved strands to achieve such breathability, or the breathability can be achieved in any other per se conventional manner.

As described above, the section 144 and/or 213 can form a free-hanging thong piece between the wearing user's skin and the leak-proof body 120. The thong piece can be relatively laterally narrow, such as at the most 50 mm or even at the most 40 mm, whereas the leak-proof body 120 can be relatively laterally wide, such as at least 40 mm. The thong piece can be at least 15 mm, such as at least 20 mm, or even at least 25 mm, laterally wide. In some embodiments, the leak-proof body 120 is at least 1.5 times or twice as laterally wide as the thong piece, at least at a narrowest part of the leak-proof body 120. Such lateral widths can be measured at the gracilis of the standing user wearing the protective garment 100.

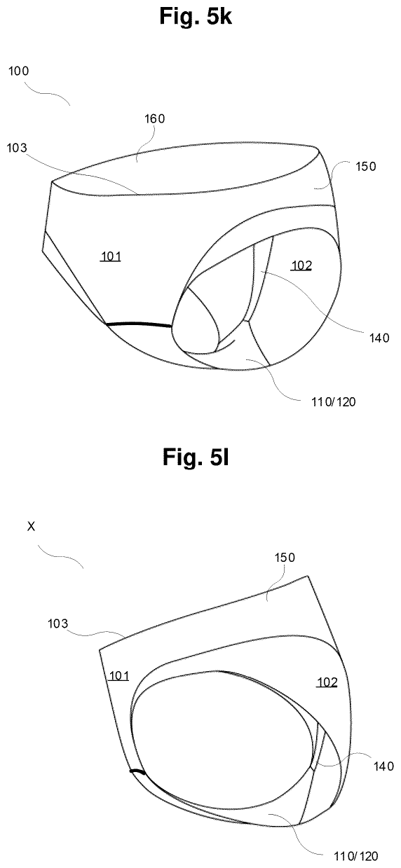

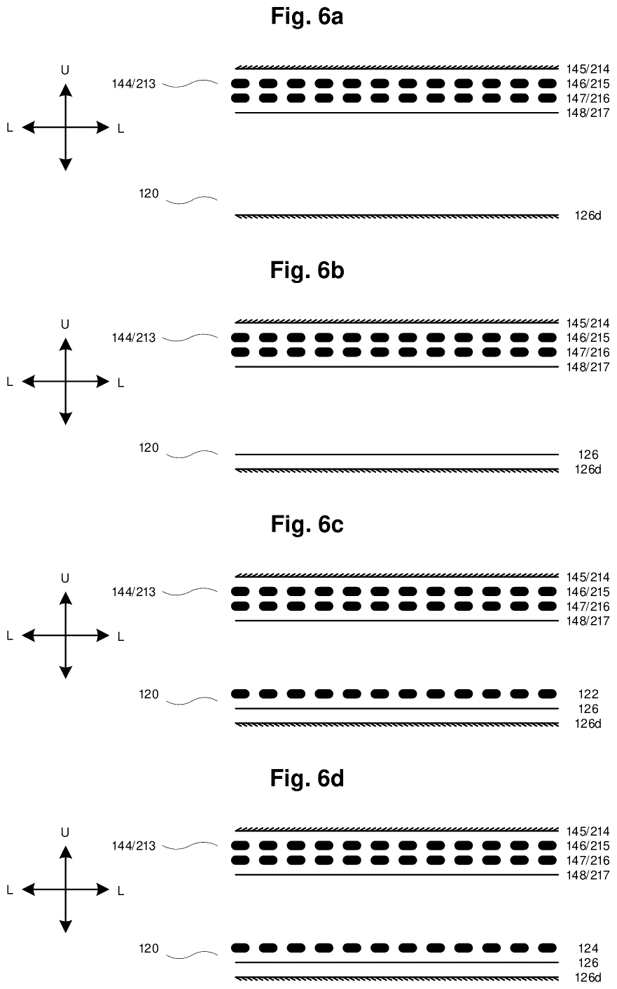

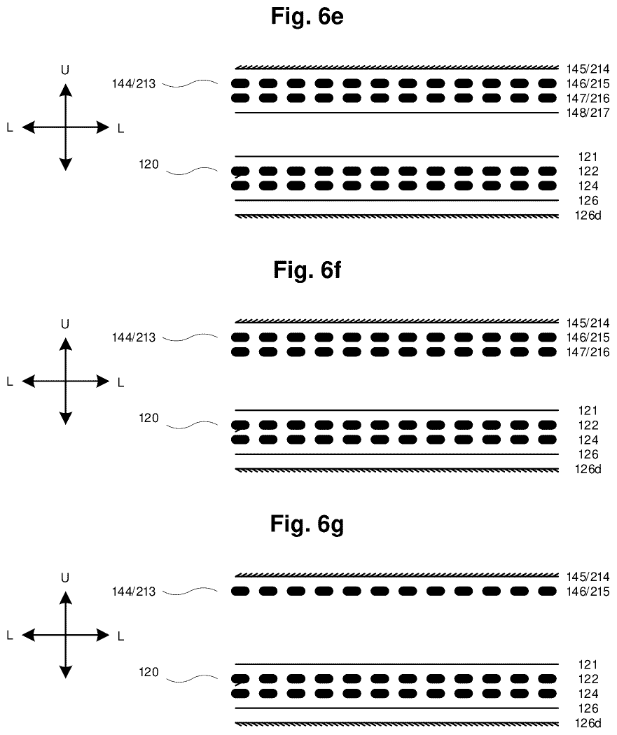

There are various ways of providing liquid-proofness of the protective garment 100 using such free-hanging thong piece. FIGS. 6a-6g show a number of possible combinations of material layers in an at least partly free-hanging thong part (i.e. the section 144 or 213 of the ventral shaping element 210 or the dorsal shaping element 130 running across the leak-proof body 120) and of the leak-proof body 120 itself.

The thong piece can be fastened to the leak-proof body 120 in different ways and in different locations on the leak-proof body 120. In some embodiments, the thong piece is fastened to the leak-proof body 120 at the dorsal end edge 120a of the leak-proof body 120. In some embodiments, the inflection point (the perineum) of the standing female user wearing the protective garment 100. In some embodiments, the thong piece is not fastened to the leak-proof body 120 dorsally of this inflection point. In some embodiments, the thong piece is fastened at the ventral end edge 120b of the leak-proof body 120. In these and other cases, the thong piece can be attached along the side edges 120c of the leak-proof body 120, such as attached to the side edges 120c ventrally of the inflection point/perineum. Ventrally of a dorsally-most fastening point between the thong piece and the leak-proof body, the thong piece can be wider than dorsally of such a fastening point.

The various layers shown in FIGS. 6a-6g can be joined together to form section 144/213 and leak-proof body 120, respectively, in various ways, including heat-pressing, welding (such as ultrasonic welding, gluing (such as bonding), and/or stitching. The joining together of the layers can be provided continuously or in a pattern across a surface plane of the layers in question and/or along a peripheral end line of the layers in question. Different or same joining techniques can be used for different pairs of layers.

The variants shown in FIGS. 6a-6g are shown as examples along a scale where FIG. 6a, at one extreme end of the scale, is arranged to provide relatively more absorption in the thong part and FIG. 6g, at the opposite extreme end, is arranged to provide relatively more absorption in the leak-proof body 120.

It is emphasized here that, as has already been described above, the section 144/213 moves freely in relation to the leak-proof body 120, apart from at a location (such as at the dorsal 120a or ventral 120b end of the leak-proof body 120) where the section 144/213 is joined together to the leak-proof body 120. In particular, such freedom of movement is present in both the lateral and vertical directions. When the standing user wears the protective garment 100, however, the section 144/213 will be drawn upwards, due to the first tension force F1 of the first element part 140 forcing the first element part 140 into the intergluteal cleft of the user; whereas the leak-proof body 120 can be more or less movable, and be fitted more or less firmly against the user depending on how the protective garment 100 is constructed.

In FIGS. 6a-6g, 145 denotes an inner surface layer of the dorsal shaping element 130, and in particular of the first element part 140, further particularly of the section 144. 214 denotes an inner surface layer of the ventral shaping element 210, and in particular of section 213. Such inner surface layer can be in the form of a liquid-permeable textile material, that can be provided with a comfortable surface texture, such as by using flocking with for instance silicon or fibers. The inner surface layer 145, 214 is generally optional.

146 and 215 denote a respective wicking layer of section 144, 213. Such wicking layer can be porous, and arranged to distribute liquid across and/or into and/or through the section 144, 213 in question.

147 and 216 denote a respective absorbing layer of section 144, 213. Such absorbing layer can comprise washable material arranged to absorb liquid, such as a so-called super-absorbent (see below).

148 and 127 denote a respective liquid barrier layer of section 144, 213. Such liquid barrier layer can, for instance, be formed by a plastic film or other suitable material that is non-permeable to liquids. The liquid barrier layer can, however, be arranged to be permeable to vapour.

121 denotes a surface layer of the leak-proof body 120. Layer 121 can be of the general type described above in relation to layers 145/214, with or without a smooth surface (such as a flocked surface). The surface layer 121 is generally optional.

122 denotes a wicking layer of the leak-proof body 120, that can be as generally described above in relation to the wicking layers 146/215.

124 denotes an absorbing layer of the leak-proof body 120, that can be as generally described above in relation to the absorbing layers 147/216.

126 denotes a barrier layer of the leak-proof body 120, that can be as generally described above in relation to the barrier layers 148/217.

126d denotes an outer surface layer of the leak-proof body 120, that can also be an outer layer of the crotch part 110, and even of the protective garment 100. The outer layer 126d can be provided so as to achieve an aesthetically pleasing surface impression, and may for instance be formed of woven or non-woven textile material.

Hence, FIG. 6a shows an embodiment in which the section 144/213 comprises, in order from the skin of the user and outwards, the inner surface layer 145/214, arranged to abut directly against the skin of the wearing user; the wicking layer 146/215, arranged to distribute liquid across and/or into the absorbing layer 147/216, arranged to absorb the liquid received via the upper layers. The section 144/213 in FIG. 6a also comprises the barrier layer 148/217, arranged to prevent absorbed liquid to distribute outwards/downwards from the section 144/213. In this embodiment, the section 144/213 is arranged to receive and absorb all liquid from the user. The leak-proof body 120 only comprises the outer layer 126d. The outer layer 126d can provide some liquid-barrier and/or absorbing properties, but in general the leak-proof body 120 in this particular embodiment receives its leak-proof function as a result of it being arranged externally to the section 144/213. For some use cases, such as for light-bleeding users, the present inventors have realized that this embodiment delivers sufficient liquid-proofness.

In the embodiment shown in FIG. 6b, the barrier layer 126 has been added to the leak-proof body 120, as a safety measure.

In the embodiment shown in FIG. 6c, the wicking layer 122 has also been added to the leak-proof body 120. In the embodiment shown in FIG. 6d, the absorbing layer 124 has instead been added to the leak-proof body 120.

In the embodiment shown in FIG. 6e, both the wicking 122 and absorbing layers have been added to the leak-proof body 120, in addition to the (optional) surface layer 121. At this point, the leak-proof body 120 is capable of receiving and absorbing relatively large amounts of liquid, in addition to the section 144/213.

In the embodiment shown in FIG. 6f, the leak-proof body 120 is arranged to receive and absorb the majority of the liquid from the user. The leak-proof body 120 features both the (optional) inner surface layer 121, the wicking layer 122, the absorbing layer 124 and the barrier layer 126. On the other hand, the section 144/213 does not include the barrier layer 148/217. Instead, the liquid is free to spread outwards from the section 144/213, to and into the leak-proof body 120.

In the embodiment shown in FIG. 6g, the absorbing layer 147/216 of section 144/213 has also been removed so that liquid in the section 144/213 is only distributed across and/or through the section 144/213, for further transport to and into the leak-proof body 120.

Generally, the section 144/213 can comprise at least one of the corresponding liquid-absorbing layer 147, 216 and the corresponding wicking layer 146, 215; and/or the section 144/213 can comprise at least the corresponding liquid-barrier layer 148, 217.

The pressing into the intergluteal cleft of the first element part 140 can achieve that the first element part 140 is deformed into a V-shape in a horizontal cross-section when the protective garment 100 is worn by the standing user. The pressing in can generally be achieved by the back part 102 pulling on the dorsal shaping element 130, and the dorsal shaping element 130, by it being elastic or inelastic, and/or stiff, not deforming/stretching (along a dorsal-ventral line) as much as the fabric of the back part 102 (and possibly also the crotch part 110) surrounding the dorsal shaping element 130, resulting in a locally applied dorsally-ventrally directed tensile force, in turn forcing first element part 140 inwards into the intergluteal cleft of the user as described. It is realized that, in the case in which the dorsal shaping element 130 is elastic, it is arranged to stretch as a result of the pulling upwards of the waist 103, but to a lesser extent than the surrounding back part 102 material, resulting in a bulging inwards of the garment 100, into the intergluteal cleft in the way described. In some embodiments, the dorsal shaping element 130 may be prestressed, such as by provided with local fabric wrinkles in combination with an elastic elongated structure urging the dorsal shaping element 130 to compress in the ventral-dorsal direction in the median plane of the standing user wearing the protective garment, or by providing the fabric of the protective garment 100 in the local vicinity to the dorsal shaping element 130, as well as the dorsal shaping element 130 itself, as an elastic fabric, achieving the corresponding effect.

It is noted that the dorsal shaping element 130 is arranged to provide properties of the protective garment 100 in the immediate vicinity of the dorsal shaping element 130 that differ from properties of the protective garment 100 at a distance from the dorsal shaping element 130 in order to achieve that the first element part 140 of the dorsal shaping element 130 is forced upwards-inwards when the waist 103 is pulled upwards as described herein.

Hence, the dorsal shaping element 130 is arranged so that an upwards pulling force applied to the waist is translated, via the back part 102, to an upwards/ventral pulling force applied to the dorsal shaping element 130, so that the first element part 140 is pressed into the intergluteal cleft of the user wearing the garment 100. Due to its connection to the crotch part 110, the first tension force F1 along the first element part 140 can also force at least part of the crotch part 110, and in particular the leak-proof body 120, to deform, such as into a saddle shape, following the local curvature of the skin of the user.

To accomplish this, the dorsal shaping element 130 is connected to the crotch part 110 in any of the ways described above. To this end, the dorsal shaping element 130 may constitute, be comprised in or comprise a joining area 133, joining together the leak-proof body 120, such as a liquid-absorbing layer 124 and/or a liquid barrier layer 126 comprised in the leak-proof body 120, to the back part 102. The section 144/213 running across the leak-proof body 120 can also be fastened to the leak-proof body 120 at the same joining area 133.

The joining area 133 can be provided in the form of a seam, a silicon bond or infusion, a weld, or any other suitable joining structure interconnecting all involved layers of material.

That the protective garment 100 is a washable garment arranged for repeated use means that materials constituting the garment 100 described herein are preferably wettable and dryable in a reversible fashion, and that all such materials are preferably permanently fastened rather than loosely arranged inside compartments or similar.

In some embodiments, the first element part 140 has a length of at least 3 cm, such as at least 5 cm, running along the intergluteal cleft of the standing user wearing the protective garment 100.

The above-discussed side parts 200 may form lateral side edges of the ventral shaping element 210, depending on if the ventral shaping element 210 is arranged as a separate layer or as an integrated part of the crotch part 110 and/or the front part 101. The side parts 200 can be provided in one of the ways described above, i.e. as a surface-deposited pattern or line of silicone or other adhesive; as a seam with particular properties; as a flocked pattern or line; as a joint between two layers; etc. The side parts 200 can be arranged to extend on either lateral side of the labia of the standing user wearing the protective garment 100, ventrally from the dorsal shaping element 130. In other words, the side parts 200 can extend along a respective lateral side of the labia, each such side edge achieving an increased tension in the material of the garment 100 along the (generally ventral-dorsal) extension line of each such side part 200, locally forcing the garment 100 upwards in relation to the standing user wearing the garment 100. In particular, the crotch part 110, such as the leak-proof body 120, such as the liquid-absorbing layer 124, and in particular the side parts 200 surrounding the labia of the user, can be pressed upwards this way.

In particular, the side parts 200 can be arranged so that, when a ventral/upward force is applied to them via the front part 101 as a result of the waist 103 being pulled upwards in relation to the user wearing the protective garment 100, it is in turn forced upwards.

Since the side parts 200 force the garment upwards on either side of the labia, but not across the labia, a very snug fit is achieved while not compromising comfort. This is particularly the case when combining the side parts 200 with the dorsal shaping element 130 of the above-described type since the combination will provide a snug fit around the whole genitals of the female user. This is particularly true in the preferred case in which the dorsal shaping element 130 is directly connected to the side parts 200, such as is illustrated in FIG. 1b. This can advantageously be achieved by arranging the first element part 140 together with the side parts 200 to together form a “Y”-shape shown in FIG. 1a. Such a combination of the dorsal shaping element 130 and the side parts 200 will distribute the above-discussed increased tension forces along the whole line from the user's intergluteal cleft to a respective point along or ventrally past the labia of the user, efficiently sealing the crotch part 110, and in particular the leak-proof body 120, against the skin of the user. It is understood that such a configuration can provide a forced saddle-shape to leak-proof body 120.

As is also illustrated in FIG. 1b, the side parts 200 can connect to either side of the ventral end edge 110b of the crotch part 110. In addition or as an alternative thereto, and as is illustrated in FIG. 1b, the side parts 200 may connect to a respective side edge 110c of the crotch part 110, such as to a ventral end point 110d of such a lateral side edge 110c. Correspondingly, the connection may also or instead be to a ventral end edge 120b and/or such a ventral side edge 120c, such as to a ventral end point of such a side edge 120c, of the leak-proof body 120 and/or to a ventral end edge and/or side edge, such as to a ventral end point of such a side edge, of a liquid-absorbing layer 124 comprised in the leak-proof body 120. This provides a good liquid seal to the parts of the crotch part 110 intended to prevent liquids from leaking out from the garment 100.

As is also illustrated in FIG. 1b, the crotch part 110, the leak-proof body 120 (and/or the liquid-absorbing layer 124, not shown in FIG. 1b) comprised in the crotch part 110 and more particularly comprised in the leak-proof body 120, can comprise a respective ventral end edge 110b; 120b, generally laterally elongated and having a convex shape softly bulging ventrally/upwards in relation to the standing user wearing the protective garment 100.

Such a bulge provides improved comfort and fit, while not jeopardizing leak-proof security. The present inventor has discovered that the bulge of the convex shape provides particularly good fit if extending at least 3 cm, ventrally/upwards, along the surface of the garment 100, in relation to the standing user wearing the protective garment 100.

As is furthermore illustrated in FIG. 1b, the crotch part 110, the leak-proof body 120 and/or the liquid-absorbing layer 124 can also comprise a respective generally laterally elongated dorsal end edge 110a; 120a. This dorsal end edge 110a, 120a can then comprise a direction change, at the intergluteal cleft of the standing user wearing the protective garment 100, the direction change defining an acute angle pointing upwards/dorsally in relation to the user.

The first element part 140 can extend dorsally and upwards 1 cm, such as at least 2 cm, such as at least 3 cm, dorsally from the apex of the direction change. Furthermore, the first element part 140 can extend at the most 30 cm, such as at the most 20 cm, such as at the most 15 cm, dorsally from the apex of the direction change.

An interconnected overlap between the first element part 140 and at least one of the crotch part 110 and the leak-proof body 120 can be between 1-30, such as 1-20 cm, of length in the ventral-dorsal direction.

As is seen in FIG. 1b, the above-mentioned joining area 133 can furthermore be arranged at the dorsal end edge of the leak-proof body 120.

The leak-proof body 120 can have a laterally local narrowest part 120d arranged between the legs, such as at or near the gracilis in a ventral/dorsal direction, of the standing user wearing the protective garment 100.

Then, as is illustrated in FIGS. 1a-1e, the side edges 120c of the leak-proof body 120 and/or of the liquid-absorbing layer 124, and/or of the central part of the leak-proof body 120, can diverge laterally outwards from the laterally narrowest part 120d, preferably in both dorsal and ventral directions. This provides a very good fit, comfort and leak-proofness, in particular in combination with the dorsal 130 and/or ventral 210 shaping elements of the types described herein.

In particular, it has proven advantageous that the side edges 120c then both extend between the dorsal end edge 120a of the leak-proof body 120 and the ventral end edge 120b of the leak-proof body 120. The side edges 120c may then be pulled ventrally/upwards by the side parts 200, while a midpoint at the dorsal end edge 120a is pulled dorsally/upwards by the dorsal shaping element 130, achieving a saddle-shape of the leak-proof body 120.

As mentioned above, each of the liquid-absorbing layers 124, 216 can be arranged to absorb a liquid, preferably in a manner so that it can hold the liquid without releasing it even if subjected to mild pressure. In contrast thereto, a spacer, open-pore foam or wicking material, such as of layers 122, 146, can hold a liquid but, like a sponge, will again release the liquid if pressed upon, sometimes even at low pressures. Hence, the liquid-absorbing layers 124, 216 can typically bind the liquid more strongly, for instance using a chemical bond. In preferred embodiments, the one or several liquid-absorbing layers 124, 216 comprise so-called “super-absorbing” materials, such as a super-absorbing polymer (“SAP”) material, forming a hydrogel when exposed to water. Preferably, such super-absorbing material is arranged in the form of a super-absorbing fiber material. In washable applications, the fibers may be manufactured from a material that does not dissolve but maintains structural integrity when exposed to water and as a result swells, such fibers being permanently bound to a flexible substrate of the liquid-absorbing layer 124, 216 in question. Super-absorbing fibers useful for the present purposes may also be contained inside a fabric container, such as a permeable mesh barrier. For instance, known super-absorbing materials comprise water absorbent, water insoluble, polymeric materials manufactured by polymerizing a water-soluble monomer or monomer blend, such as acrylic acid, in the presence of a polyethylenically unsaturated monomer, such as N,N′methylenebisacrylamide. See, for instance, US2011172621 A1 for more information about super-absorbing materials.

When such fiber materials are washed and dried, they release any absorbed bodily fluids and return to a state in which they are again prepared to absorb liquids anew.

Such super-absorbing fiber containing material will be a washable woven or non-woven material, such as airlaid, thermo-carded, needle-punch, netting or similar. Washable open-pore foam structures can also be used.

The wicking layers 122, 215 can be made from such as so-called spacer materials/spacer fabrics. This is a material that has been manufactured to contain voids, forming an open-pore structure that absorbs, holds and transports liquids through capillary forces, much like a sponge. For instance, such spacer materials can be made as stitched textiles. By controlling the stitching in 3D space, such spacer material may be manufactured to display open-pore structures having different properties. Such wicking/spacer/distribution materials are sold by, for instance, Muller Textiles, Troy, MI, USA. See EP 2938310 B1 for examples of such distribution materials.

Unless stated otherwise, all materials described herein and making up the protective garment 100 are flexible materials.

Above, preferred embodiments have been described. However, it is apparent to the skilled person that many modifications can be made to the disclosed embodiments without departing from the basic idea of the invention.

It is understood that the various aspects described herein relate to the design and construction of a leak-proof garment which is washable for repeated use, providing superior comfort and that can be designed and constructed to be aesthetically pleasing. Therefore, it is apparent that the various aspects can be combined in many different ways, depending on the detailed requirements and goals, to achieve one or several of these objects.

For instance, using dorsal and/or ventral shaping elements of the present type of the present type, provides a very tight and/or snug fit to the wearing user. Using a leak-proof body with various layers of the types described herein, and/or a dorsal and/or ventral section running across the leak-proof body, provides a very good leak-protection and absorption. Any combination between any aspect of the former group of aspects with any aspect of the latter group of aspects will achieve a garment which has a tight fit and also good leak protection. Since leak-protection is dependent both on a tight fit and an efficient handling of leaking liquids in and in connection to the leak-proof body, there are many synergistic effects by performing such combinations. The laterally separated shaping elements 190 allow for a smaller absorbing surface, in particular in combination with the various embodiments of the leak-proof body 120 and possibly also the free-hanging thong part as described above.

Moreover, it is realized that the Figures and examples provided herein are only for illustration of the aspects, and that a protective garment according to the present invention can include many additional parts in addition to the ones described herein, such as ornamental or functional parts.

In addition to the various layers described herein, the garment 100 may furthermore contain one of more layers of a textile net, non-flocked fibers and/or a foam. For instance, an internal layer, directly contacting the skin of the user wearing the garment 100, may comprise an internal textile net or foam material. In other examples, textile layers of different elasticity may be used, for instance to form the dorsal 130 and/or ventral 210 shaping elements as a middle elastic textile layer, defining the shape in question, arranged between two less elastic textile layers. In other examples, the grain direction of different layers of woven fabric may be varied to achieve the desired properties, such as locally achieving a less elastic or even stiff area defining the dorsal shaping element 130 by arranging an enclosed piece of woven fabric there having a grain direction selected to provide relative stiffness in the dorsal-ventral direction.

Hence, the invention is not limited to the described embodiments, but can be varied within the scope of the enclosed claims.

1. A protective garment in the form of a pantie, a pair of trousers or other legwear with or without an upper/torso part, comprising:
a front part;
a back part;
a waist; and
a crotch part having a leak-proof body,
the protective garment being a washable garment arranged for repeated use,
wherein:
the protective garment comprises a dorsal shaping element, in turn comprising a flexible first element part having a ventral end and a dorsal end, a flexible second element part and a flexible third element part;
the first element part is arranged to run along the intergluteal cleft of a standing user wearing the protective garment;
the second and third element parts are each arranged to extend laterally across the waist and/or buttocks of the standing user wearing the protective garment, towards a respective hip of the standing user;
the protective garment is arranged so that, as a result of the standing user wearing the protective garment, the first element part applies a first tension force along the intergluteal cleft of the user whereas the second element part applies an at least partly lateral second tension force and the third element part applies an at least partly lateral third tension force;
the dorsal shaping element is arranged as a separate, overlapping, flexible layer in relation to the rest of the protective garment;
the dorsal shaping element is non-connected to, and freely movable in relation to, the protective garment at least between the crotch part and a fastening point to the rest of the protective garment; and
the dorsal shaping element is fastened to the crotch part and/or runs internally to the crotch part when the protective garment is worn by the standing user.

2. The protective garment of claim 1, wherein the first element part, at its ventral end, is fastened to or constitutes an integrated part of the leak-proof body.

3. The protective garment of claim 1, wherein:
the dorsal shaping element is non-connected to, and freely movable in relation to, the protective garment at least between the leak-proof body and the fastening point to the rest of the protective garment; and
the dorsal shaping element is fastened to the leak-proof body and/or runs internally to the leak-proof body when the protective garment is worn by the standing user.

4. The protective garment of claim 1, wherein the second element part and the third element part are both arranged to run laterally to a respective iliac crest of the standing user wearing the protective garment.

5. The protective garment of claim 1, wherein the protective garment further comprises a pair of laterally separated shaping elements, each running ventrally from the first element part, in relation to the standing user wearing the protective garment, along either side of the leak-proof body, to a position ventrally of the perineum of the standing user, each of the laterally separated shaping elements being arranged to apply a tension force along its length when the protective garment is worn by the standing user.

6. The protective garment of claim 5, wherein the laterally separated shaping elements are arranged to extend on either lateral side of the labia of the standing user wearing the protective garment, ventrally from the first element part, and arranged so that, when a ventral/upward force is applied to the front part and the back part as a result of the waist being pulled upwards in relation to the user wearing the protective garment, they are in turn forced upwards.

7. The protective garment of claim 5, wherein:
the dorsal shaping element is directly connected to the laterally separated shaping elements and the dorsal shaping element, together with the laterally separated shaping elements, forms a “Y”-shape.

8. The protective garment of claim 1, wherein the dorsal shaping element is fastened to the protective garment at the waist of the protective garment.

9. The protective garment of claim 1, wherein the second element part and the third element part are both fastened to the protective garment at a respective point ventrally to the iliac crest of the standing user wearing the protective garment, so that each of the second element part and the third element part runs, from the dorsal end of the first element part, around the iliac crest of the standing user wearing the protective garment and again laterally towards but not up to a front center line of the front part.

10. The protective garment of claim 1, wherein the dorsal shaping element is fastened to a ventral end of the leak-proof body, and runs over the leak-proof body, internally to the leak-proof body when the protective garment is worn by the standing user, between a dorsal end of the leak-proof body and the ventral end of the leak-proof body.

11. The protective garment of claim 10, wherein a section of the dorsal shaping element running across the leak-proof body is tapered so that a dorsal end of the section is narrower than a ventral end of the section, in relation to the standing user wearing the protective garment.

12. The protective garment of claim 10, wherein a section of the dorsal shaping element running across the leak-proof body is laterally narrower than the leak-proof body.

13. The protective garment of claim 10, wherein at least the section of the dorsal shaping element running across the leak-proof body comprises at least one of a liquid-absorbing layer and a wicking layer.

14. The protective garment of claim 13, wherein at least the section of the dorsal shaping element running across the leak-proof body comprises a liquid-barrier layer.

15. The protective garment of claim 1, wherein:
the protective garment comprises a ventral shaping element;
the ventral shaping element is fastened to the leak-proof body; and
the ventral shaping element is fastened to the protective garment at the waist of the protective garment but is non-connected to the protective garment between the waist and the leak-proof body, or the ventral shaping element is connected to the dorsal shaping element near the waist.

16. The protective garment of claim 15, wherein the ventral shaping element is fastened to the leak-proof body at a dorsal and/or ventral end of the leak-proof body.

17. The protective garment of claim 16, wherein at least a section of the ventral shaping element running across the leak-proof body comprises at least one of a liquid-absorbing layer and a wicking layer.

18. The protective garment of claim 17, wherein at least the section of the ventral shaping element running across the leak-proof body comprises a liquid-barrier layer.

19. The protective garment of claim 1, wherein the first element part is fastened to the leak-proof body at the perineum of the standing user wearing the protective garment.

20. The protective garment of claim 1, wherein the dorsal shaping element constitutes, is comprised in or comprises a joining area, joining together the leak-proof body, such as a liquid-absorbing layer comprised in the leak-proof body, to the back part.
